(19) United States
(12) Patent Application Publication
(10) Pub. No.: US 2025/0213546 A1
(43) Pub. Date: Jul. 3, 2025

(54) TREATING CANCER WITH SMALL MOLECULES THAT INHIBIT ACSS-2

(71) Applicant: DREXEL UNIVERSITY, Philadelphia, PA (US)

(72) Inventors: Alexej DICK, Philadelphia, PA (US); Simon COCKLIN, Santa Fe, NM (US); Adel Ahmed Rashad AHMED, Fishers, IN (US); Mauricio J. REGINATO, Merion Station, PA (US); Lorela CIRAKU, Philadelphia, PA (US); Emily M. ESQUEA, Philadelphia, PA (US)

(21) Appl. No.: 18/851,289

(22) PCT Filed: Mar. 30, 2023
(86) PCT No.: PCT/US2023/065148

§ 371 Date: Sep. 26, 2024

Related U.S. Application Data
(60) Provisional application No. 63/326,363, filed on Apr. 1, 2022.

(51) Int. Cl. A61K 31/451; A61K 31/40; A61K 31/437; A61K 31/445; A61K 31/454; A61K 31/496; A61K 31/5377; A61P 35/04

(52) U.S. Cl. CPC A61K 31/451; A61K 31/40; A61K 31/437; A61K 31/445; A61K 31/454; A61K 31/496; A61K 31/5377; A61P 35/04

(57) ABSTRACT

Cancers develop specific metabolic adaptations such as the opportunistic utilization of acetate as an alternative carbon source and the generation of acetyl-CoA mediated by ACSS-2, which is upregulated in response to nutrient and hypoxic stress. This pathway can fuel various processes involving acetyl-CoA, including de novo fatty acid synthesis and protein acetylation. Provided herein are methods of treating a cancerous disease in a subject using compounds that inhibit ACSS-2.

### CROSS-REFERENCE TO RELATED APPLICATIONS

This application claims priority to U.S. Provisional Application Ser. No. 63/326,363, filed on Apr. 1, 2022, the disclosure of which is hereby incorporated by reference in its entirety.

### GOVERNMENT RIGHTS

This invention was made with government support under AI150491 awarded by the National Institutes of Health and UO1 CA244303 awarded by the National Cancer Institute. The government has certain rights in the invention.

### TECHNICAL FIELD

Disclosed herein are methods of treating a cancerous disease in a subject with compounds that inhibit ACSS-2.

### BACKGROUND

Breast cancer is the second greatest cause of brain metastasis after lung cancer, and breast cancer brain metastases (BCBM) develop if cells spread from breast tumors to the brain. Brain metastasis is not curable, and >80% of patients die within a year of diagnosis. Moreover, traditional therapeutics, chemotherapy, and radiation induce undesirable effects in healthy tissue, significantly reducing the quality of the patient's life. The harsh nutrient-depleted and hypoxic microenvironment forces cancer cells with their high metabolic demand to adapt metabolically and evolve to these challenging conditions. Adaptation, however, induces resistance to chemo/immunotherapy and radiation that can cause further malignant progression. Tumor-specific metabolic adaptations evolve as new and promising targets for cancer treatment. One such cancer-specific metabolic adaptation is the opportunistic utilization of acetate as an alternative carbon source and the generation of acetyl-CoA. Cancer cells utilize this pathway to fuel various processes involving acetyl-CoA, including de novo fatty acid synthesis and protein acetylation.

A functional genomic screen identified acyl-coenzyme A synthetase short-family member 2 (ACSS-2) as vital for tumor growth during hypoxic stress. ACSS-2 is upregulated in response to nutrient and hypoxic stress within the tumor microenvironment. The nucleocytosolic enzyme converts acetate to acetyl-CoA in an ATP-dependent fashion under hypoxic and low nutrient conditions. Furthermore, ACSS-2 was shown to influence gene transcription through two critical types of acetylation, namely acetylation of specific histones and acetylation of transcription factors. Acetylation, therefore, affects metabolic reprogramming and cell cycle progression in cancer via epigenetic means.

Reducing ACSS-2 levels can block cancer cell growth both in vitro and in vivo. Genetically targeting ACSS-2 decreased the growth of various cancers, including breast, melanoma, liver, prostate, myeloma, and glioblastoma, highlighting the vital role of acetate metabolism in cancer. The central role and importance of ACSS-2 in various cancer types highlights the enormous potential for novel, safe, and effective cancer treatments. Disclosed herein are methods for the pharmacological inhibition of the human ACSS-2 enzyme with novel chemotypes that have superior computationally predicted drug-like properties.

### SUMMARY

Disclosed herein are methods for treating a cancerous disease in a subject, comprising administering to the subject a therapeutically effective amount of any one or more of compounds described herein which inhibit acyl-coenzyme A synthetase short-family member 2.

### BRIEF DESCRIPTION OF THE DRAWINGS

The summary, as well as the following detailed description, is further understood when read in conjunction with the appended drawings. For the purpose of illustrating the disclosed methods, there are shown in the drawings exemplary embodiments of the methods; however, the methods are not limited to the specific embodiments disclosed. In the drawings:

FIG. 1 illustrates the computational pipeline for the discovery of AD-2441 and its analogs. AD-2441 and analogs display superior predicted drug-like properties (StarDrop). The in silico prediction of drug-like metrics was achieved using StarDrop 6.4 (Optibrium, Ltd., Cambridge, UK), implementing the oral central nervous system (CNS) drug profile, supplemented with an additional parameter for log D.

FIG. 2A, FIG. 2B, FIG. 2C, and FIG. 2D illustrate the computational prediction of drug-like properties of AD-2441 and its analogs. FIG. 2A shows oral CNS Scoring profile score vs. log S of AD-2441 and its analogs including two currently known control inhibitors VY-3-249 and VY-3-135. FIG. 2B depicts the P-glycoprotein (Pgp) category and predicted probability of AD-2441 and analogs. FIG. 2C depicts the predicted plasma protein binding (90% threshold, PPB90) category and predicted probability. FIG. 2D displays the predicted blood-brain barrier (BBB) distribution/category and category-probability of AD-2441 and analogs.

FIG. 3A, FIG. 3B, FIG. 3C, and FIG. 3D illustrate the computational prediction of metabolic stability of AD-2441 and its analogs. FIG. 3A shows the prediction of the major metabolizing CYP isoforms for AD-2441 and analogs. The majority of compounds are predicted to be metabolized by the 3A4 isoform, except AD-1363 and AD8972, which are predicted to be metabolized by the 2D6 isoform. FIG. 3B displays a lower boundary for predicted 3A4 affinity of AD-2441 and analogs using the hydrogen bond and dehydration scoring function (HYDE) implemented in SeeSAR11.2. FIG. 3C shows the overall composite site lability (CSL) score and number of labile sites (for metabolism) for AD-2441 and analogs. A lower CSL score indicates a more stable molecule. The prediction was achieved using the StarDrop (version 7) P450 module. FIG. 3D displays the 2D6 affinity category of AD-2441 and analogs as predicted by the Stardrop P450 module.

FIG. 4A and FIG. 4B illustrate where AD-2441 and its analogs are predicted to bind ACSS-2 within the CMP binding pocket.

FIG. 5A, FIG. 5B, and FIG. 5C illustrate molecular dynamics simulations of AD-2441 and its analogs bound to ACSS-2 (PDB code: 5JRH).

FIG. 6A and FIG. 6B illustrate molecular interaction patterns of AD-2441 and its analogs with ACSS-2 (PDB code: 5JRH) based on MD simulation over 10 ns.

FIG. 7A, FIG. 7B, and FIG. 7C illustrate the computational prediction of drug-like properties of AD-2441 and its analogs. FIG. 7A displays the drug-like properties of AD-2441 and its analogs including two currently known control inhibitors VY-3-249 and VY-3-135. Prediction of the major metabolizing CYP isoform for all compounds. The majority of compounds are predicted to be metabolized by the 3A4 isoform, except AD-1363 and AD8972, which are metabolized (predicted) by the 2D6 isoform. FIG. 7B shows the CYP isoform coloring in FIG. 7A. Column 2 (P45) indicates the number of labile or stable atoms within the molecule. A lower score indicates a more stable molecule. The prediction was achieved using the StarDrop (version 7) P450 module. FIG. 7C displays the oral CNS scoring profile calculation and significance of each drug-like property used for calculation. A higher score indicates improved drug-like properties. VY-3-249 (control inhibitor) and VY-3-135 were used as controls (DOI: 10.1158/0008-5472.CAN-20-1847).

FIG. 8A and FIG. 8B illustrate representative sensorgrams for AD-2441 and its analogs binding to hACSS-2. Lines represent collected data from the dilution series, whereas black lines signify the fits to a 1:1 binding model-interaction parameters derived from a triplicate (n=3) of data given in Table 2.

FIG. 9 illustrates binding isotherms of AD-2441 and analogs to immobilized hACSS-2. Binding isotherms are derived from the data in FIG. 4A and FIG. 4B. Experiments were performed in triplicate, and data displayed with standard deviations (SD with n=3).

FIG. 10A, FIG. 10B, FIG. 10C, FIG. 10D, FIG. 10E, FIG. 10F, and FIG. 10G illustrate in vitro and ex vivo potency evaluation of AD-2441. FIG. 10A demonstrates the effect of AD-2441 on BCBM cells (MDA-MB-231-BR cells) in vitro using a Crystal Violet Assay. Detection 48 hrs post-treatment with AD-2441. FIG. 10B shows quantification of cell viability (MDA-MB-231-BR cells) using an MTT viability assay, n=4, p<0.05. FIG. 10C shows representative images of stained colonies formed in the clonogenic assay after 10-14 days from MDA-MB-231BR cells treated with a control or ACSS2 inhibitors. Two-way ANOVA with Dunnett's test reported as mean±SEM, ***p<0.001, ****p<0.0001. FIG. 10D depicts ex vivo mouse brain slice model for evaluation of 4T1-BR tumor growth (bioluminescence intensity) post-treatment with AD-2441 and corresponding quantification (n=1) (FIG. 10E). FIG. 10F depicts ex vivo mouse brain slice model for evaluation of MDA-MB-231-BR tumor growth (volume and bioluminescence intensity) post-treatment with AD-2441 and corresponding quantification (n=4 for day 0-day 6 and n=2 for day 6-day 10) (FIG. 10G). VY-3-249 was used as a control (DOI: 10.1158/0008-5472.CAN-20-1847). FIG. 10H shows representative images of stained colonies formed in the clonogenic assay after 10-14 days from MDA-MB-231BR cells following treatment with a control or the indicated ACSS2 inhibitor. Two-way ANOVA with Dunnett's test reported as mean±SEM, **p<0.01, ***p<0.001. FIG. 10I shows bioluminescence imaging of luciferase tagged MDA-MB-231BR tumor-brain slices over 6-day period treated with DMSO or the indicated ACSS2 inhibitor. Two-way ANOVA with Dunnett's test reported as mean±SEM, **p<0.01. FIG. 10J is a graph quantifying brain slice viability after a 6-day treatment period with DMSO or the indicated ACSS2 inhibitor after incubation in MTS solution. Negative control was incubated in PFA for 1 hour prior to MTS. Two-way ANOVA with Dunnett's test reported as mean±SEM, **p<0.01.

FIG. 11A and FIG. 11B illustrates a reduction in tumor growth by close and distant chemical analogs of AD-2441. FIG. 11A demonstrates that analogs reduce BCBM (MDA-MB-231-BR cells) tumor growth in vitro using a Crystal Violet Assay. Detection 48 hrs post-treatment with 20 μM and 10 of the compound. FIG. 11B shows the quantification of BCBM tumor growth reduction in FIG. 11A (n=3). FIG. 11C shows representative images of MDA-MB-231BR cells grown in a monolayer and treated with increasing doses of the indicated ACSS2 inhibitor analog for 24 hours and stained with crystal violet.

FIG. 12A, FIG. 12B, FIG. 12C, and FIG. 12D illustrate ex vivo potency evaluation of AD-2441 analogs. FIG. 12A depicts an ex vivo mouse brain slice model for evaluation of MDA-MB-231-BR tumor growth (bioluminescence intensity) post-treatment with AD-2441 analogs at 100 μM and corresponding quantification (n=1) (FIG. 12B). FIG. 12C depicts ex vivo mouse brain slice model for evaluation of MDA-MB-231-BR tumor growth (bioluminescence intensity) post-treatment with AD-2441 analogs at 200 μM and corresponding quantification (n=1) (FIG. 12D). VY-3-249 was used as a control (DOI: 10.1158/0008-5472.CAN-20-1847).

FIG. 13A, FIG. 13B, and FIG. 13C illustrate that AD-2441 alters the expression level of FASN and possibly reduces de novo fatty acid synthesis. FIG. 13A displays a diagram depicting ACSS-2 providing acetyl-CoA for numerous metabolic processes vital for cancer proliferation. The human fatty acid synthase (FASN) is essential for BCBM growth and survival. FIG. 13B shows that hACSS-2 does regulate FASN expression levels via histone acetylation in healthy and tumor microenvironments. FIG. 13C demonstrates that AD-2441 downregulates FASN expression levels and possibly reduces de novo lipid synthesis, essential for BCBM tumor growth. VY-3-249 was used as a control (DOI: 10.1158/0008-5472.CAN-20-1847).

FIG. 14 shows ACSS2 enzyme activity measured in the presence of the indicated compounds.

FIG. 15A and FIG. 15B demonstrate that ACSS2 Inhibitor Analogs induce apoptosis in BCBM cells. FIG. 15A shows flow cytometry plots of MDA-MB-231BR cells treated with DMSO or the indicated ACSS2 inhibitor for 24 hours. FIG. 15B quantifies the fraction of apoptotic cells from MDA-MB-231BR treated with DMSO or the indicated ACSS2 inhibitor for 24 hours. Two-way ANOVA with Dunnett's test reported as mean±SEM, **p<0.01.

FIG. 16A, FIG. 16B, and FIG. 16C demonstrate that ACSS2 inhibitor analogs reduce levels of lipid droplet storage. FIG. 16A shows immunoblots of MDA-MB-231BR cell lysates after infection with lentivirus containing shSCR or shACSS2 and immunoblots of MDA-MB-231BR cells lysates after treatment with ACSS2 inhibitors for 48 hours. FIG. 16B illustrates micrographs of 231BR cells treated with the indicated ACSS2 inhibitor and stained for lipid droplets. FIG. 16C is a graph quantifying the number of lipid droplets in cells treated with a control or the indicated ACSS2 inhibitor.

FIG. 17A shows bioluminescence images of luciferase tagged MDA-MB-231BR tumor-brain slices over 6-day period following treatment with DMSO or the indicated ACSS2 inhibitor with and without 6gY irradiation. FIG. 17B is a graph quantifying bioluminescence of luciferase tagged MDA-MB-231BR tumor-brain slices over 6-day period following treatment with DMSO or the indicated ACSS2 inhibitor with and without 6gY irradiation.

FIG. 18A shows bioluminescence images of luciferase tagged MDA-MB-231BR tumor-brain slices over 6-day period following treatment with DMSO or the indicated ACSS2 inhibitor with and without 6gY irradiation. FIG. 18B is a graph quantifying bioluminescence of luciferase tagged MDA-MB-231BR tumor-brain slices over 6-day period following treatment with DMSO or the indicated ACSS2 inhibitor with and without 6gY irradiation.

FIG. 19A depicts a graphical workflow depicting IP injections of ACSS2 inhibitor analogs, blood extraction and brain removal for liquid chromatography-mass spectrometry (LCMS). FIG. 19B shows the LCMS quantification of the indicated compound.

FIG. 20A shows bioluminescence images of luciferase tagged MDA-MB-231BR following intracranial injection into nude mice and treatment with 20 mg/kg of vehicle or with the 8007 ACSS2 inhibitor. FIG. 20B is a graph quantifying bioluminescence over time from FIG. 20A. FIG. 20C is a graph illustrating the weight of mice over time treated with treated with the 8007 ACSS2 inhibitor from FIG. 20A.

FIG. 21A shows bioluminescence images of luciferase tagged MDA-MB-231BR following intracranial injection into nude mice and treatment with 20 mg/kg of vehicle or with the 5584 ACSS2 inhibitor. FIG. 21B is a graph quantifying bioluminescence over time from FIG. 21A. FIG. 21C is a graph illustrating the weight of mice over time treated with the 5584 ACSS2 inhibitor from FIG. 21A.

FIG. 22A shows bioluminescence images of luciferase tagged 4T1BR following intracranial injection into balb/c mice and treatment with 20 mg/kg of vehicle or the indicated ACSS2 inhibitor. FIG. 22B is a graph quantifying bioluminescence over time from FIG. 22A. FIG. 22C is a graph illustrating weight of mice over time treated with the indicated ACSS2 inhibitor from FIG. 22A. FIG. 22D shows bioluminescence images of luciferase tagged 4T1BR following intracranial injection into balb/c mice and treated with 20 mg/kg of vehicle or with the 5584 ACSS2 inhibitor. FIG. 22E is a graph quantifying bioluminescence over time from FIG. 22D.

### DETAILED DESCRIPTION OF ILLUSTRATIVE EMBODIMENTS

The disclosed methods may be understood more readily by reference to the following detailed description taken in connection with the accompanying figures, which form a part of this disclosure. It is to be understood that the disclosed methods are not limited to the specific methods described and/or shown herein, and that the terminology used herein is for the purpose of describing particular embodiments by way of example only and is not intended to be limiting of the claimed methods.

Unless specifically stated otherwise, any description as to a possible mechanism or mode of action or reason for improvement is meant to be illustrative only, and the disclosed methods are not to be constrained by the correctness or incorrectness of any such suggested mechanism or mode of action or reason for improvement.

It is to be appreciated that certain features of the disclosed methods which are, for clarity, described herein in the context of separate embodiments, may also be provided in combination in a single embodiment. Conversely, various features of the disclosed methods that are, for brevity, described in the context of a single embodiment, may also be provided separately or in any subcombination.

As used herein, the singular forms “a,” “an,” and “the” include the plural.

Various terms relating to aspects of the description are used throughout the specification and claims. Such terms are to be given their ordinary meaning in the art unless otherwise indicated. Other specifically defined terms are to be construed in a manner consistent with the definitions provided herein.

The term “comprising” is intended to include examples encompassed by the terms “consisting essentially of” and “consisting of”; similarly, the term “consisting essentially of” is intended to include examples encompassed by the term “consisting of.”

“Inhibited” growth (e.g., referring to cells, such as tumor cells) refers to a measurable decrease in in vitro or in vivo cell growth upon contact with the compound or combination thereof when compared to the growth of the same cells in the absence of the compound or combination thereof. Inhibition of growth of a cell in vitro or in vivo may be at least about 10%, about 20%, about 30%, about 40%, about 50%, about 60%, about 70%, about 80%, about 90%, about 99%, or about 100%. Inhibition of cell growth can occur by a variety of mechanisms, for example by antibody-mediated ADCC, ADCP and/or CDC, apoptosis, necrosis, or by inhibition of cell proliferation.

The term “about” when used in reference to numerical ranges, cutoffs, or specific values is used to indicate that the recited values may vary by up to as much as 10% from the listed value. Thus, the term “about” is used to encompass variations of +10% or less, variations of ±5% or less, variations of ±1% or less, variations of ±0.5% or less, or variations of ±0.1% or less from the specified value.

“Treat,” “treatment,” and like terms refer to both therapeutic treatment and prophylactic or preventative measures, and includes reducing the severity and/or frequency of symptoms, eliminating symptoms and/or the underlying cause of the symptoms, reducing the frequency or likelihood of symptoms and/or their underlying cause, and improving or remediating damage caused, directly or indirectly, by the cancerous disease. Treatment also includes prolonging survival as compared to the expected survival of a subject not receiving treatment. Subjects to be treated include those that have the condition or disorder as well as those prone to have the condition or disorder or those in which the condition or disorder is to be prevented.

As used herein, “administering to the subject” and similar terms indicate a procedure by which the compounds are ingested or injected into a subject such that target cells, tissues, or segments of the body of the subject are contacted with the compounds.

As used herein, the phrase “therapeutically effective amount” refers to an amount of the compound or combination thereof, as described herein, effective to achieve a particular biological or therapeutic result such as, but not limited to, biological or therapeutic results disclosed, described, or exemplified herein. The therapeutically effective amount may vary according to factors such as the disease state, age, sex, and weight of the individual, and the ability of the composition to cause a desired response in a subject. Exemplary indicators of a therapeutically effect amount include, for example, improved well-being of the patient, reduction of a tumor burden, arrested or slowed growth of a cancer, and/or absence of metastasis of cancer cells to other locations in the body.

The term “subject” as used herein is intended to mean any animal, in particular, mammals. The methods are applicable to human and nonhuman animals, although preferably used with mice and humans, and most preferably with humans. “Subject” and “patient” are used interchangeably herein.

Disclosed herein are methods for treating a cancerous disease in a subject, the method comprising administering to the subject a therapeutically effective amount of any one or more of the following compounds:

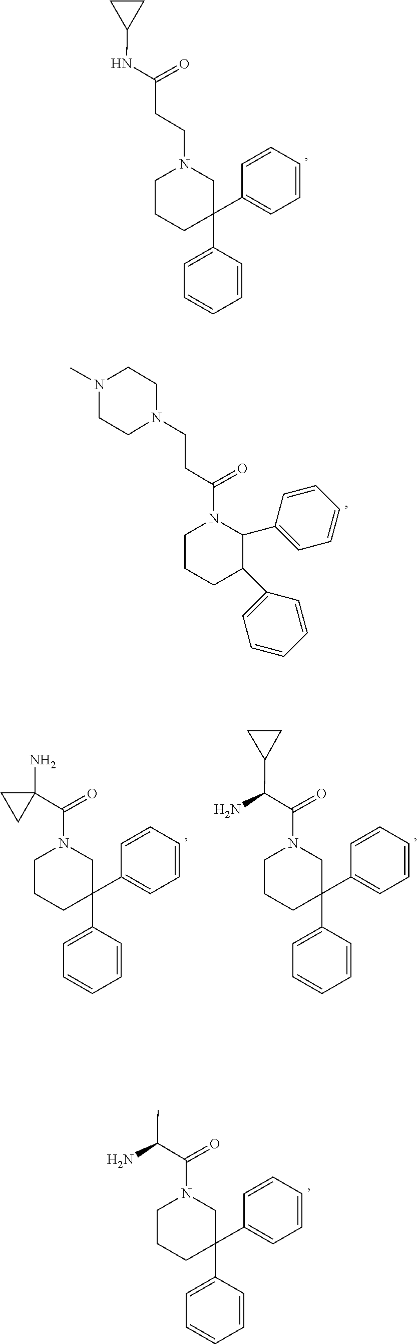 [SMILES: O=C(CCN1CCCC(c2ccccc2)(c2ccccc2)C1)NC1CC1 ; CN1CCN(CCC(=O)N2CCCC(c3ccccc3)C2c2ccccc2)CC1 ; NC1(C(=O)N2CCCC(c3ccccc3)(c3ccccc3)C2)CC1 ; N[C@H](C(=O)N1CCCC(c2ccccc2)(c2ccccc2)C1)C1CC1 ; C[C@H](N)C(=O)N1CCCC(c2ccccc2)(c2ccccc2)C1]

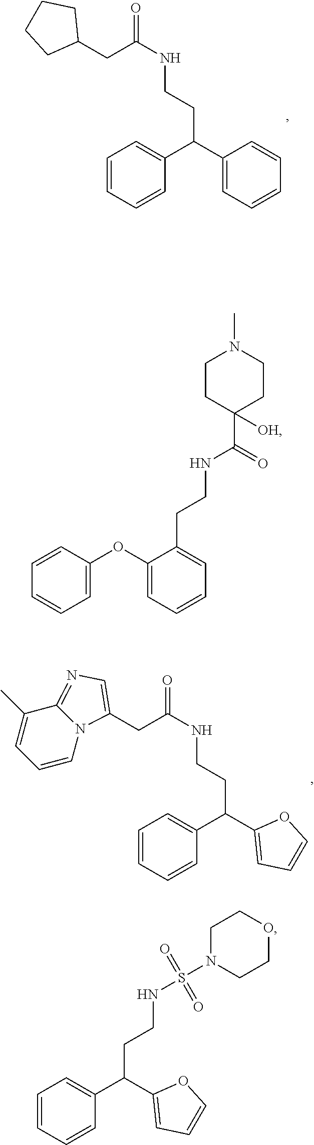 [SMILES: O=C(CC1CCCC1)NCCC(c1ccccc1)c1ccccc1 ; CN1CCC(O)(C(=O)NCCc2ccccc2Oc2ccccc2)CC1 ; Cc1cccn2c(CC(=O)NCCC(c3ccccc3)c3ccco3)cnc12 ; O=S(=O)(NCCC(c1ccccc1)c1ccco1)N1CCOCC1]

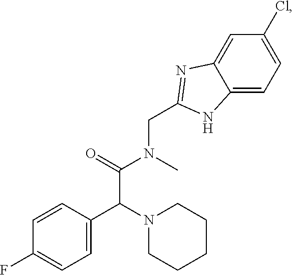 [SMILES: CN(Cc1nc2cc(Cl)ccc2[nH]1)C(=O)C(c1ccc(F)cc1)N1CCCCC1]

or a pharmaceutically acceptable salt thereof.

In some embodiments, the subject is administered one of the disclosed compounds. In other embodiments, the subject is administered a combination of the disclosed compounds. Such a combination comprises at least two of the disclosed compounds administered simultaneously. In other embodiments, the compounds, or combinations thereof, are administered at different points in time.

The disclosed methods can be used to treat subjects with any cancerous disease. In some embodiments, the cancer has increased acyl-coenzyme A synthetase short-family member 2 expression and/or activity. Suitable cancerous diseases comtemplated for treatment with the disclosed compounds include, but are not limited to, brain cancer and breast cancer. In some embodiments, the brain cancer is a brain metastasis. Suitable brain metastases include, but are not limited to, a breast cancer brain metastatic tumor.

The compounds disclosed herein are suitable for treatment of cancerous diseases of the brain. In some embodiments, one or more of the compounds cross the blood-brain barrier to exert their effect on acyl-coenzyme A synthetase short-family member 2 (ACSS-2). In other embodiments, one or more of the compounds do not cross the blood-brain barrier to exert their effect on ACSS-2.

The compounds described herein exert their effect on ACSS-2 by inhibiting or inactivating the enzyme. In some embodiments, the disclosed compounds reduce the enzymatic activity of ACSS-2. In other embodiments, the disclosed compounds inhibit the enzymatic activity of ACSS-2. In some embodiments, reduction or inhibition of the enzymatic activity of ACSS-2 results in inhibited growth of the cancerous disease.

In addition to cancer, other diseases, disorders, and conditions may be mediated or exacerbated by the activity of ACSS-2 either at baseline or elevated levels. Cancer is not the only circumstance where inhibition of ACSS-2 may result in favorable or desired outcomes. As a result, the disclosed compounds are suitable for any disease, disorder, or condition in which inhibition of ACSS-2 may produce a favorable or desired outcome. For example, genetically modified mice containing an ACSS-2 gene knockout accumulate less fat when placed on a high fat diet relative to a wild-type control. Thus, inhibition or inactivation of ACSS-2 could also be a suitable strategy to treat or combat obesity or fat accumulation. Those of skill in the art will appreciate that many diseases, disorders, and conditions may benefit from inhibition of ACSS-2 mediated by the compounds disclosed herein.

The methods of treatment comprising administration of the herein described compounds may further comprise administering radiation therapy to the subject. The radiation therapy can be directed to a tumor or a metastasis. In some embodiments, a brain metastasis irradiated. The irradiated brain metastasis can be a breast cancer brain metastatic tumor.

Also disclosed herein are methods of inhibiting and/or inactivating acyl-coenzyme A synthetase short-family member 2 (ACSS-2), the method comprising contacting the ACSS-2 with of any one or more of the following compounds:

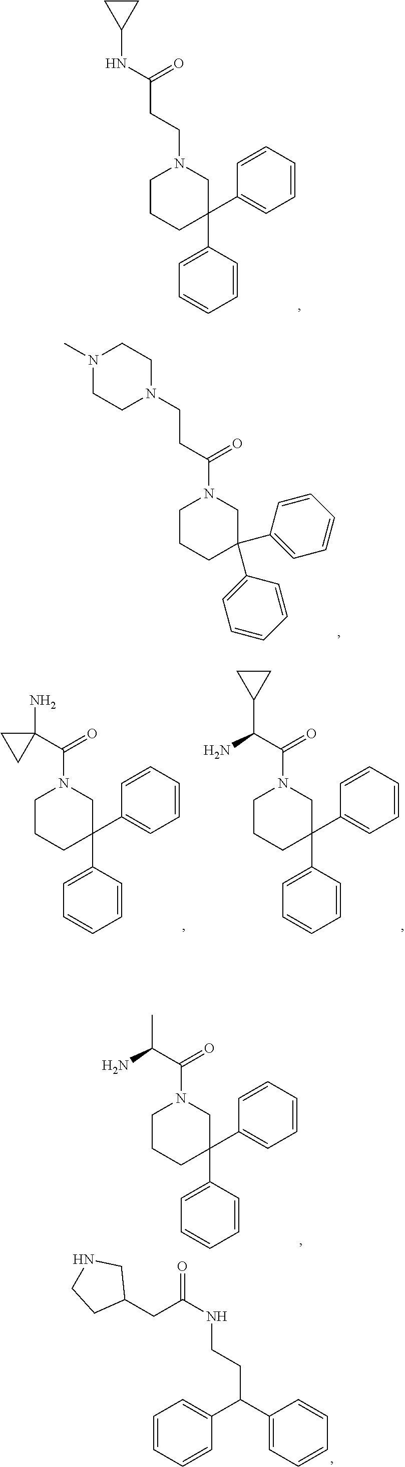

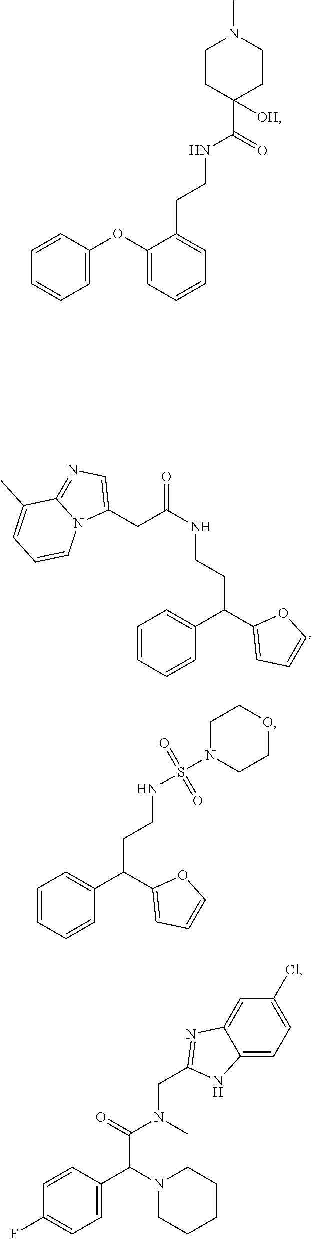 [SMILES: CN1CCC(O)(C(=O)NCCc2ccccc2Oc2ccccc2)CC1 ; Cc1cccn2c(CC(=O)NCCC(c3ccccc3)c3ccco3)cnc12 ; O=S(=O)(NCCC(c1ccccc1)c1ccco1)N1CCOCC1 ; CN(Cc1nc2cc(Cl)ccc2[nH]1)C(=O)C(c1ccc(F)cc1)N1CCCCC1]

or a pharmaceutically acceptable salt thereof. The one or more compounds can inactivate the ACSS-2. The one or more compounds can inhibit the ACSS-2. The one or more compounds can degrade the ACSS-2. The inhibition and/or inactivation of ACSS-2 can occur in vitro. Examples of in vitro inhibition and/or inactivation of ACSS-2 include, but are not limited to, inhibition and/or inactivation of purified ACSS-2, inhibition and/or inactivation of ACSS-2 within laboratory grown cells, or inhibition and/or inactivation of ACSS-2 that exists in a mixture of cellular components, such as a cell lysate. The ACSS-2 may be inhibited and/or inactivated in prokaryotic or eukaryotic cells. Inhibition and/or inactivation of ACSS-2 can result in inhibited growth or reduced metabolic rate of laboratory grown cells. Purified ACSS-2 or ACSS-2 that exists in a mixture of cellular components may be recombinant ACSS-2 or ACSS-2 that is naturally expressed. The inhibition and/or inactivation of ACSS-2 can occur in vivo. Examples of in vivo inhibition and/or inactivation of ACSS-2 include, but are not limited to, inhibition and/or inactivation of ACSS-2 within an organism or subject whose cells express ACSS-2. Inhibition and/or inactivation of ACSS-2 can result in inhibited growth or reduced metabolic rate of cells in the organism or subject.

### EXAMPLES

The following examples are provided to further describe some of the embodiments disclosed herein. The examples are intended to illustrate, not to limit, the disclosed embodiments.

### Example 1

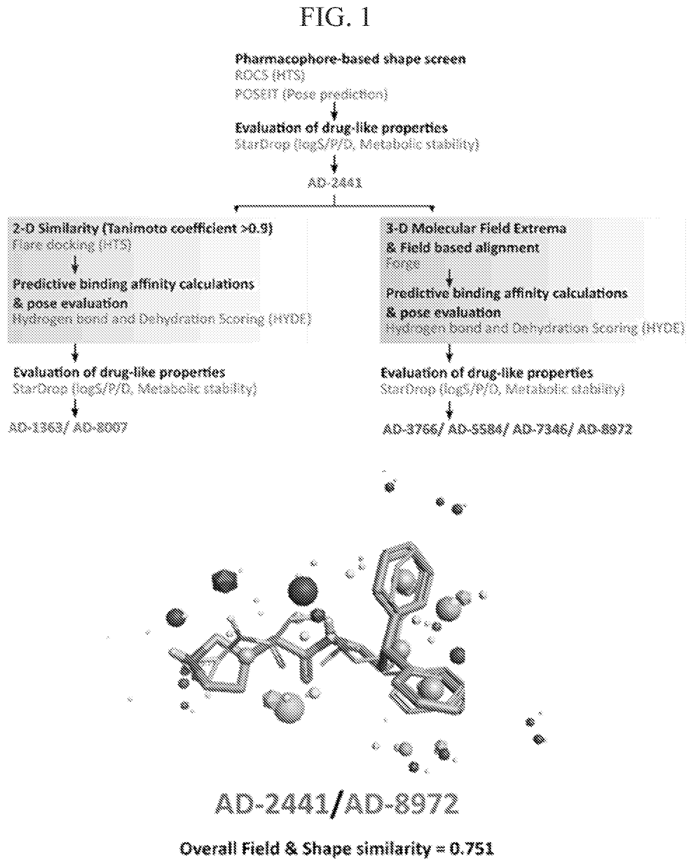

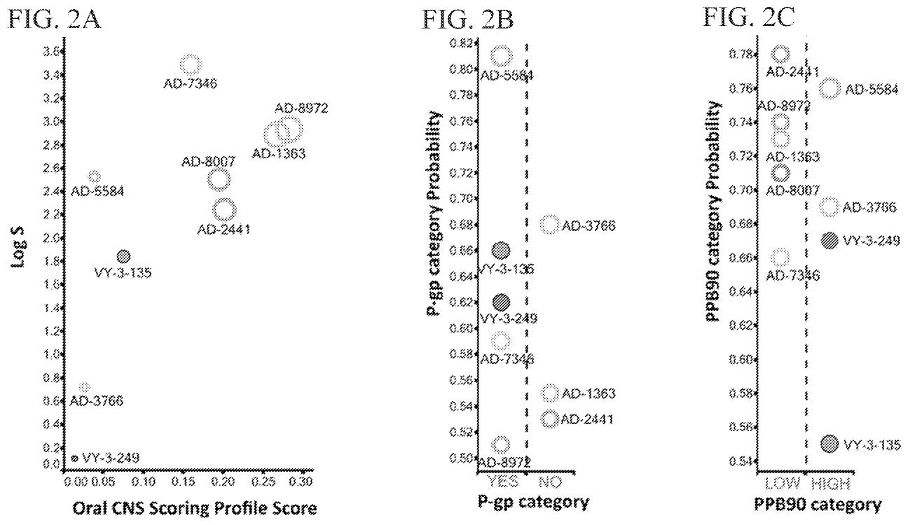

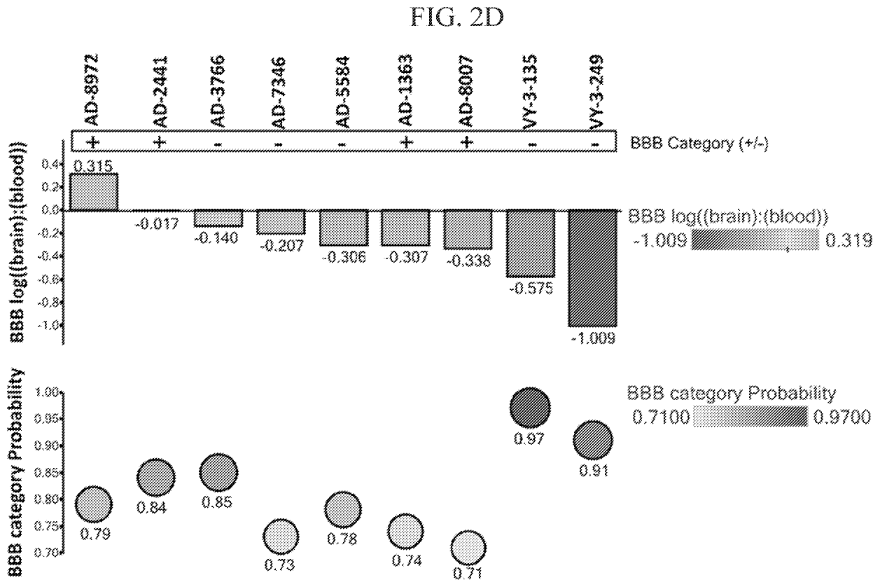

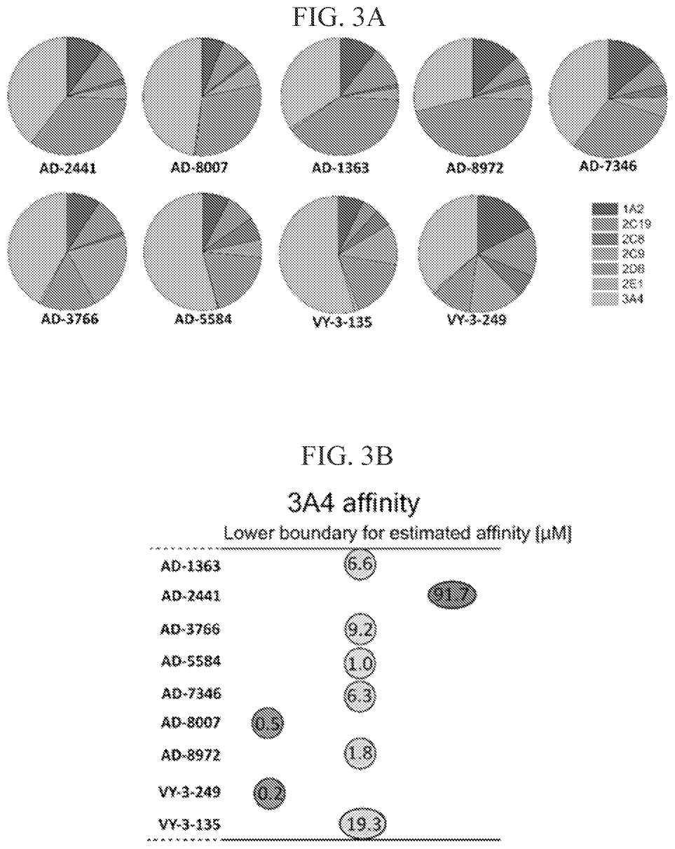

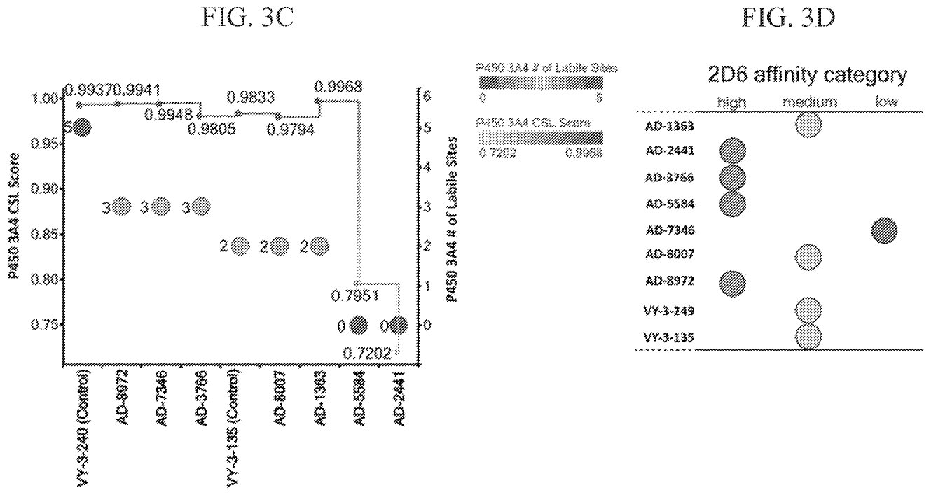

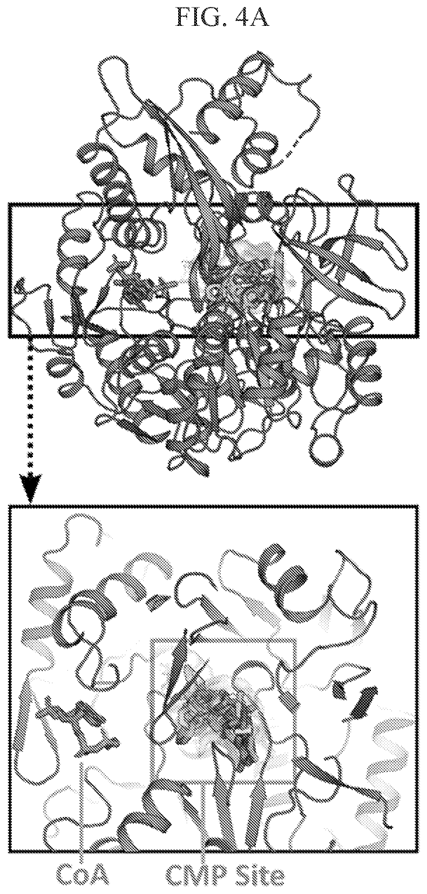

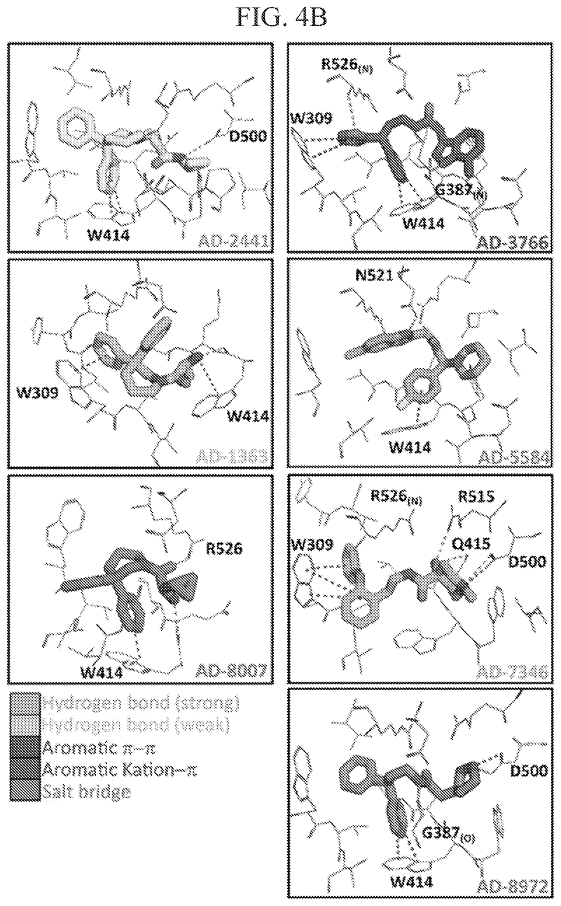

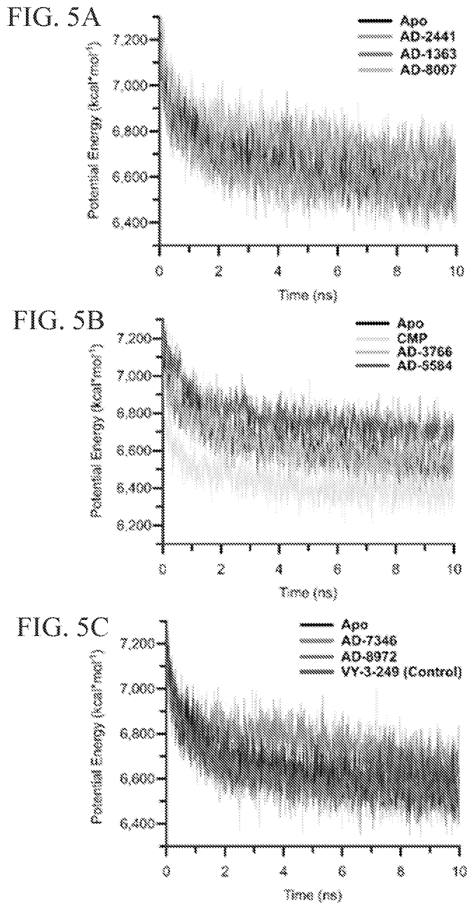

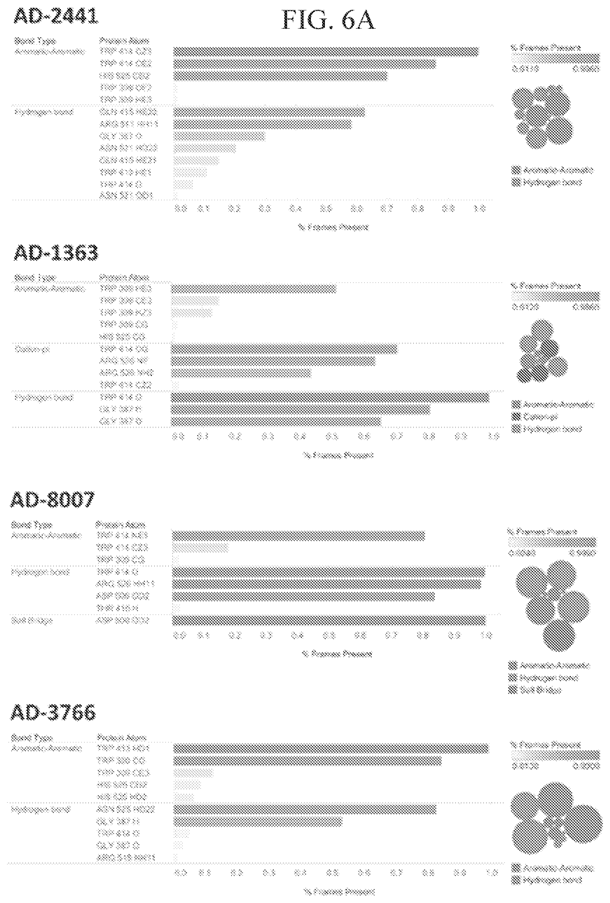

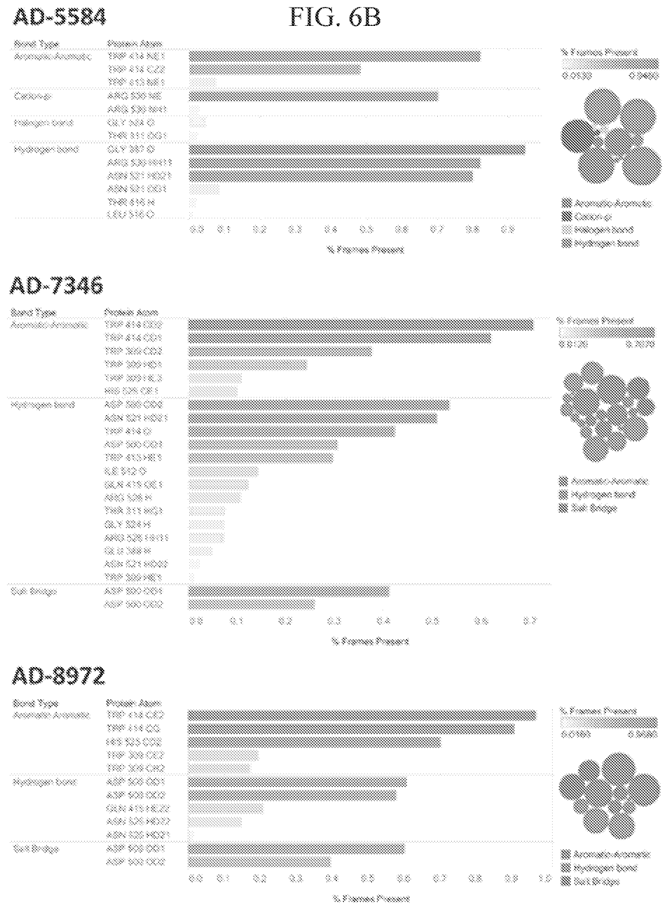

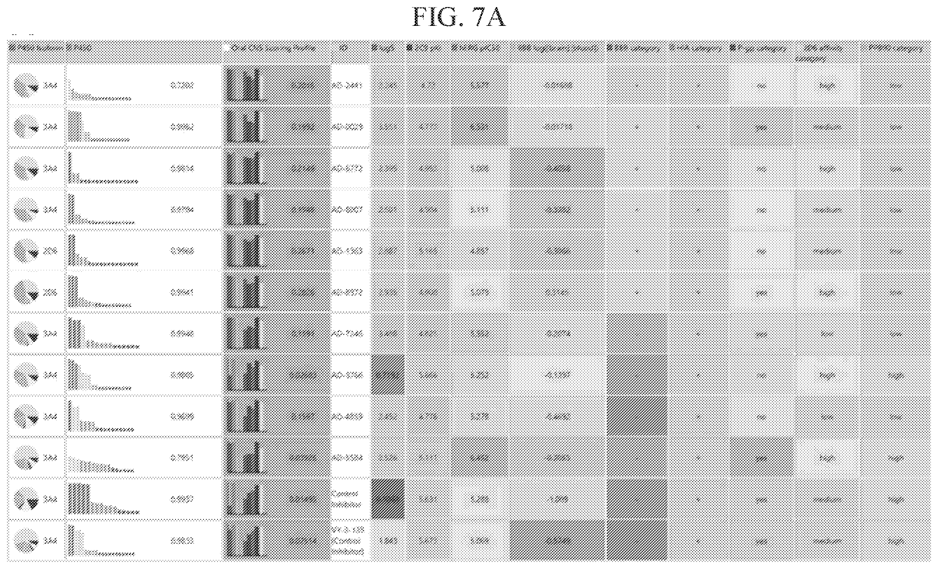

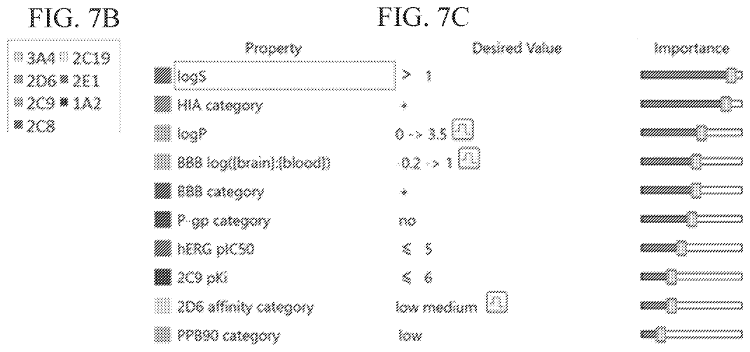

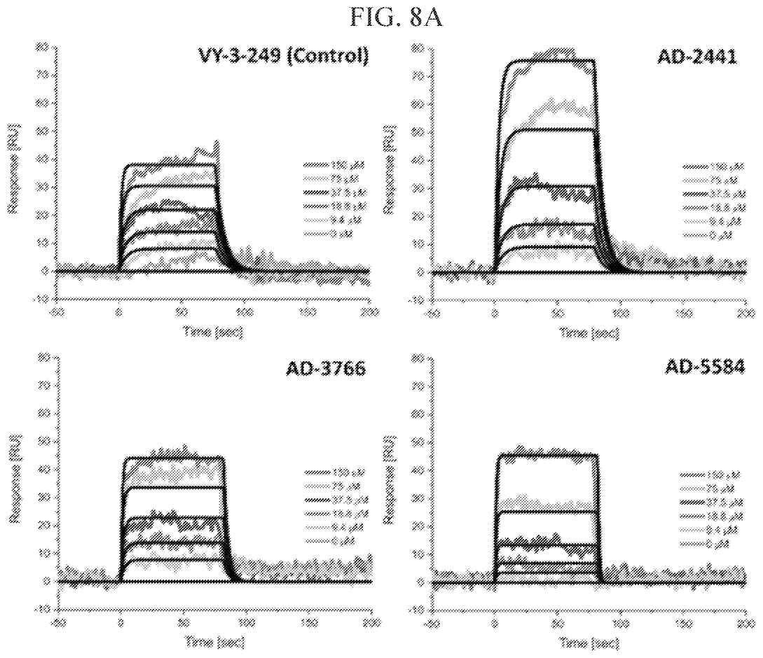

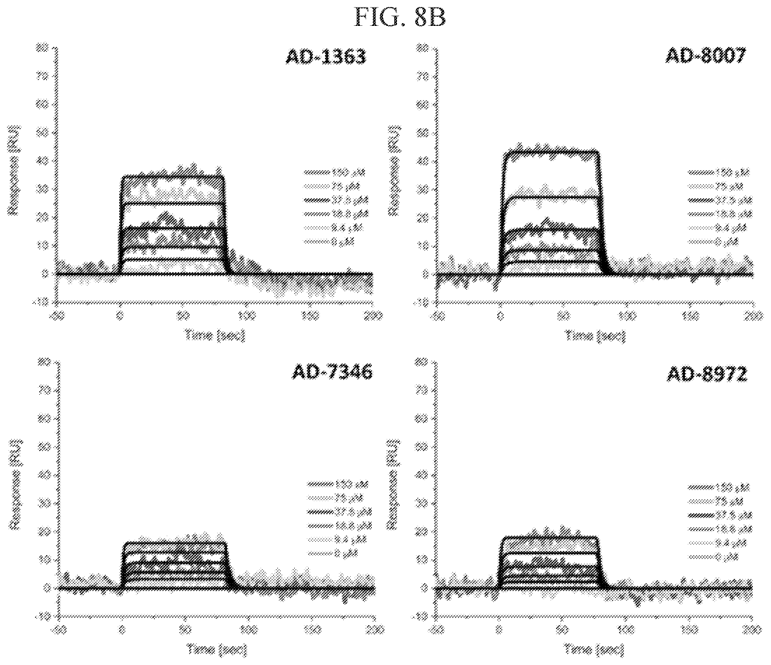

A rigorous computational screening and filtering process (FIG. 1) was used to discover 10 small molecules for pharmacological inhibition of the human ACSS-2 enzyme with novel chemotypes and superior computationally predicted drug-like properties compared to control inhibitors (FIG. 2A, FIG. 2B, FIG. 2C, FIG. 2D, FIG. 7A, FIG. 7B, FIG. 7C), most notably improved blood-brain barrier penetration, improved solubility, and metabolic stability (FIG. 3A, FIG. 3B, FIG. 3C, FIG. 3D, FIG. 7A, FIG. 7B, FIG. 7C). Overall all 10 compounds showed an improved computed oral central nervous system (CNS) score compared to the control compounds. AD-2441 and its analogs (AD-0029, AD-6772, AD-8007, AD-1363, AD-8972, AD-7346, AD-3766, AD-4859, AD-5584) were predicted to bind ACSS-2 within the CMP binding pocket (FIG. 4A, FIG. 4B). Molecular dynamics simulations exemplified the predicted molecular interactions and binding of AD-2441 and its analogs with ACSS-2 (FIG. 5A, FIG. 5B, FIG. 5C, FIG. 6A, and FIG. 6B).

Docking calculations for hACSS-2 inhibitors. Compounds were built and energy-minimized using the MM2 force field (ChemBio3D Ultra 13.0) with RMS gradient of 0.01 and number of alterations of 104. The minimized structures were then saved as pdb files for the docking simulations.

The fitting of each pose was independently corrected and validated using POSIT (OpenEye Scientific Software, Santa Fe, NM. eyesopen.com).

The hACSS-2 inhibitors were prepared and then energy minimized using Flare version 5 (Cresset®, Litlington, Cambridgeshire, UK, cresset-group.com/flare/) with a root mean squared (RMS) gradient cutoff of 0.2 kcal/mol/A and 10000 iterations. The crystal structure of ACSS-2 from Salmonella enterica (PDB code: 5JRH) was prepared using Flare, version 5 (Cresset®, Litlington, Cambridgeshire, UK, cresset-group.com/flare/) to allow protonation at pH 7.0 and removal of residue gaps and further prepared using Autodock tools, where essential hydrogen atoms, Kollman united atom type charges, and solvation parameters were added. The grid box for the docking search was centered around the CMP and CoA binding site with a spacing grid of 0.375 Å using the Autogrid program. Docking calculations were performed using AutoDock via DockingServer. Inhibitors were further energy minimized using the MMFF94 Force Field method and Gasteiger partial charges were added. Non-polar hydrogen atoms were merged, and rotatable bonds were defined. AutoDock parameter set- and distance-dependent dielectric functions were used in the calculation of the van der Waals and the electrostatic terms, respectively. Docking simulations were performed using the Lamarckian genetic algorithm (LGA) and the Solis & Wets local search method. The initial position, orientation, and torsions of the ligand molecules were set randomly. Rotatable torsion angles were released during docking. Each docking experiment was derived from 100 different runs that were set to terminate after a maximum of 2500000 energy evaluations. The population size was set to 150. During the search, a translational step of 0.2 Å, and quaternion and torsion steps of 5 were applied.

Molecular Dynamics (MD) Simulations. For comparison of AD-2441 and its analogs against the Apo form of hACSS-2, complex stability (potential energy of the complex in kcal/mol) of the generated complexes was performed using the molecular dynamics interface within Flare, version 5 (Cresset®, Litlington, Cambridgeshire, UK, cresset-group.com/flare/). The energy minimized complexes were simulated using the AMBER force field (GAFF2) over 10 ns with an implicit solvent model (OBC2) at 298.0 K. The complex was equilibrated for 200 ps and trajectory frames generated with 4.0 fs steps and a hydrogen mass repartitioning of 1.5.

### Example 2

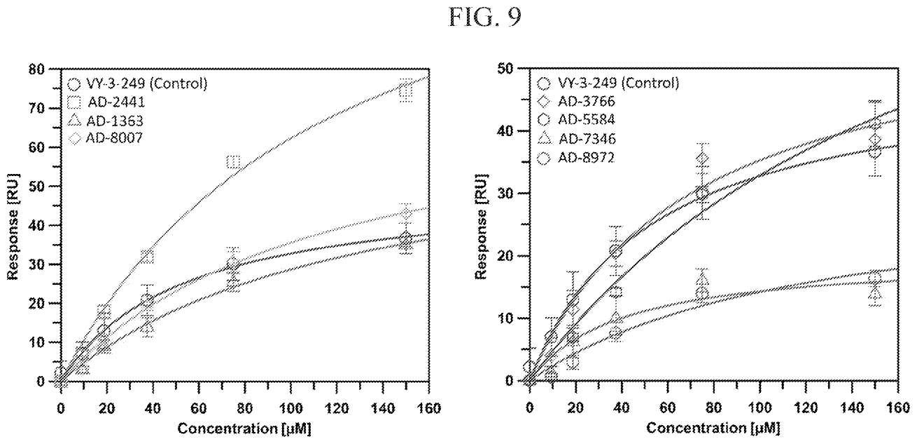

Preliminary studies show that the parental AD-2441 compound and the 9 additional analogs directly engage the hACSS-2 protein with affinities in the low μM-range using surface plasmon resonance (SPR) spectroscopy (FIG. 8A, FIG. 8B, FIG. 9, Table 1, Table 2).

SPR characterization. All binding assays were performed on a ProteOn XPR36 SPR Protein Interaction Array System (Bio-Rad Laboratories, Hercules, CA, USA). The instrument temperature was set at 25° C. for all kinetic analyses. ProteOn GLH sensor chips were preconditioned with two short pulses each (10 s) of 50 mM NaOH, 100 mM HCl, and 0.5% sodium dodecyl sulfide. Then the system was equilibrated with running buffer (1×PBS pH 7.4, 3% DMSO and 0.005% polysorbate 20). The surface of a GLH sensor chip was activated with a 1:100 dilution of a 1:1 mixture of 1-ethyl-3-(3-dimethylaminopropyl) carbodiimide hydrochloride (0.2 M) and sulfo-N-hydroxysuccinimide (0.05 M). Immediately after chip activation, the HIV-1 CA protein constructs were prepared at a concentration of 10 μg/mL in 10 mM sodium acetate, pH 5.5, and injected across ligand flow channels for 5 min at a flow rate of 30 μL/min. Then, after unreacted protein had been washed out, excess active ester groups on the sensor surface were capped by a 5 min injection of 1M ethanolamine HCl (pH 8.0) at a flow rate of 5 μL/min. A reference surface was similarly created by immobilizing a nonspecific protein (IgG b12 anti-HIV-1 gp120; was obtained through the NIH AIDS Reagent Program, Division of AIDS, NIAID, NIH: Anti-HIV-1 gp120 Monoclonal (IgG1 b12) from Dr. Dennis Burton and Carlos Barbas) and was used as a background to correct nonspecific binding. Serial dilutions of hACSS-2 inhibitors were then prepared in the running buffer and injected at a flow rate of 100 μL/min, for a 50 s association phase, followed by up to a 5 min dissociation phase using the “one-shot kinetics” capability of the ProteOn instrument. Data were analyzed using the ProteOn Manager Software version 3.0 (Bio-Rad). The responses from the reference flow cell were subtracted to account for the nonspecific binding and injection artifacts. Experimental data were fitted to a simple 1:1 binding model. Experiments were performed in triplicate to detect kinetic and equilibrium dissociation constants (KD).

Overproduction of hACSS-2 was achieved using a prokaryotic expression system. Briefly, the plasmids containing the C- and N-terminally His-tagged hACSS-2 DNA were transformed into BL21 (DE3) RIL competent cells (Agilent Technologies, Wilmington, DE) and were expressed in auto-inducing media ZYP-5052 overnight at 15° C. with shaking at 225 rpm. The bacterial expressions were then spun down, the supernatant discarded, and the pellets resuspended. After the cells were lysed via sonication, the sample was subjected to ultracentrifugation, and the clarified lysate was applied to a Talon cobalt resin affinity column (Clonetech Laboratories, Mountain View, CA). The bound protein was eluted from the column using 1×PBS pH 7.4 with 0.3 M imidazole, then dialyzed overnight into 20 mM Tris-HCl pH 8.0, concentrated to 10 mg/mL, aliquoted, and stored at −80° C.

TABLE 1
Chemical structure and affinity (K_{D}) assessment of AD-2441 and its analogs for
hACSS-2 using surface plasmon resonance spectroscopy (SPR). Experiments performed
in triplicate and representative isotherms (one site binding model) are depicted for each
compound.

| | Affinity (K_{D} in μM) for hACSS-2 |
|---|---|
| Compound ID/Structure | mean ± SD (n = 3) |
| 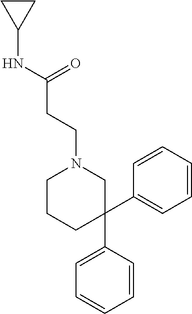 [SMILES: O=C(CCN1CCCC(c2ccccc2)(c2ccccc2)C1)NC1CC1] | 105 ± 16 |
| AD-2441 | |
| 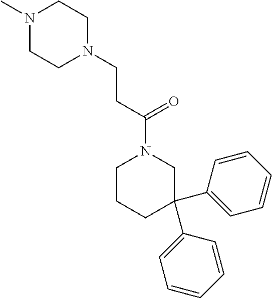 [SMILES: CN1CCN(CCC(=O)N2CCCC(c3ccccc3)(c3ccccc3)C2)CC1] | 356 ± 108 |
| AD-0029 | |
| 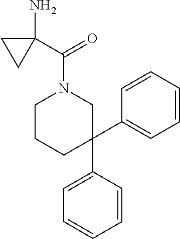 [SMILES: NC1(C(=O)N2CCCC(c3ccccc3)(c3ccccc3)C2)CC1] | 80 ± 20 |
| AD-6772 | |
| 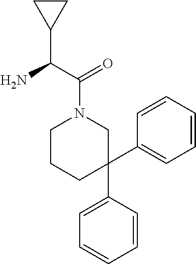 [SMILES: N[C@H](C(=O)N1CCCC(c2ccccc2)(c2ccccc2)C1)C1CC1] | 89 ± 20 |
| AD-8007 | |
| 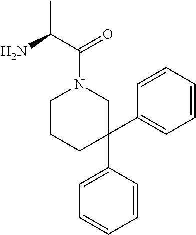 [SMILES: C[C@H](N)C(=O)N1CCCC(c2ccccc2)(c2ccccc2)C1] | 85 ± 16 |
| AD-1363 | |
| 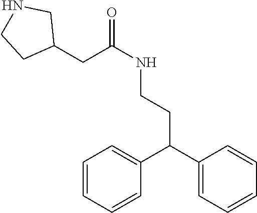 [SMILES: O=C(CC1CCNC1)NCCC(c1ccccc1)c1ccccc1] | 116 ± 82 |
| AD-8972 | |
| 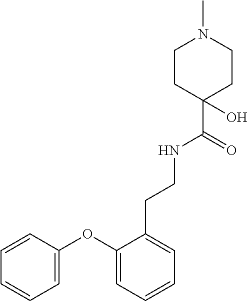 [SMILES: CN1CCC(O)(C(=O)NCCc2ccccc2Oc2ccccc2)CC1] | 53 ± 21 |
| AD-7346 | |
| 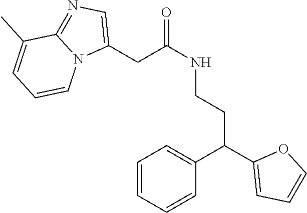 [SMILES: Cc1cccn2c(CC(=O)NCCC(c3ccccc3)c3ccco3)cnc12] | 67 ± 13 |
| AD-3766 | |
| 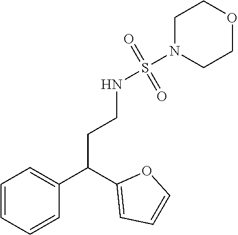 [SMILES: O=S(=O)(NCCC(c1ccccc1)c1ccco1)N1CCOCC1] | 52 ± 2 |
| AD-4859 | |
| 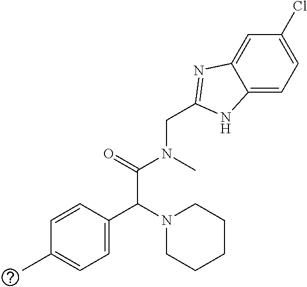 [SMILES: Cc1ccc(C(C(=O)N(C)Cc2nc3cc(Cl)ccc3[nH]2)N2CCCCC2)cc1] | 161 ± 108 |
| AD-5584 | |

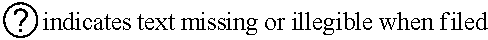

TABLE 2
Kinetic and equilibrium parameters for AD-2441 and analogs binding to immobilized hACSS-2.
Equilibrium Dissociation Constant (K_{D}) derived from a Langmuir isotherm equilibrium
fit or a global fit. Values are mean ± standard deviation (SD) with n = 3.

| Compound | K_{D} [μM] | | kₒₙ | | k_{off} | | K_{D} [μM] | |
|---|---|---|---|---|---|---|---|---|
| ID | equilibrium | ±SD | [M⁻¹s⁻¹] | ±SD | [s⁻¹] | ±SD | kinetic/global | ±SD |
| VY-3-249 | 53.2 | 7.3 | 1.80E+03 | 8.93E+02 | 1.05E−01 | 2.95E−02 | 66.0 | 18.7 |
| (Control) | | | | | | | | |
| AD-2441 | 116.9 | 21.4 | 1.46E+03 | 2.80E+02 | 1.75E−01 | 2.89E−02 | 121.3 | 14.1 |
| AD-1363 | 130.7 | 25.9 | 3.77E+03 | 1.75E+03 | 6.34E−01 | 6.86E−02 | 208.1 | 87.7 |
| AD-8007 | 116.6 | 19.5 | 4.79E+03 | 3.10E+03 | 5.47E−01 | 2.01E−01 | 149.4 | 54.6 |
| AD-3766 | 70.6 | 24.7 | 5.01E+03 | 1.73E+03 | 3.59E−01 | 5.55E−02 | 77.6 | 20.4 |
| AD-5584 | 188.7 | 82.8 | 2.02E+03 | 6.10E+02 | 6.32E−01 | 2.25E−01 | 352.3 | 180.3 |
| AD-7346 | 39.8 | 23.9 | 1.01E+04 | 5.17E+03 | 3.99E−01 | 1.11E−01 | 49.4 | 22.5 |
| AD-8972 | 124.0 | 70.5 | 2.83E+03 | 5.05E+02 | 4.01E−01 | 4.20E−02 | 149.3 | 45.0 |

### Example 3

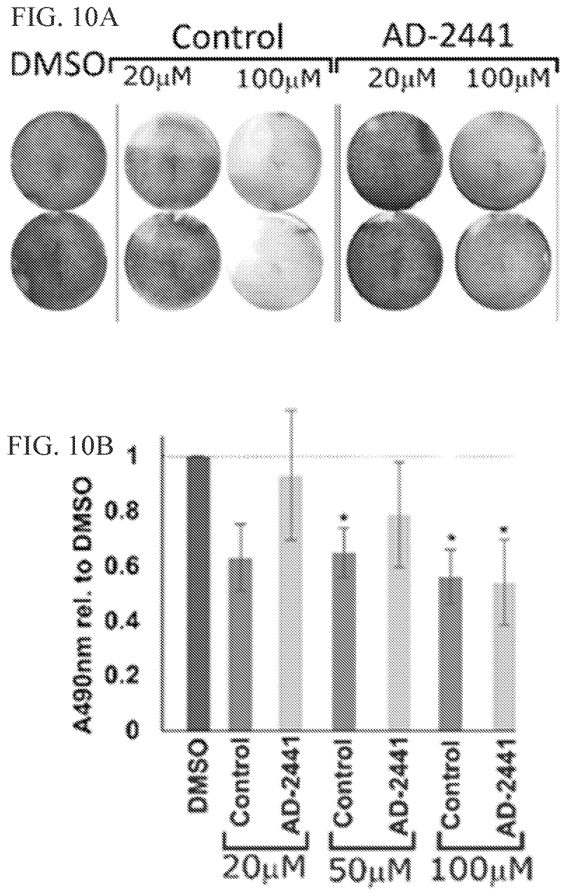

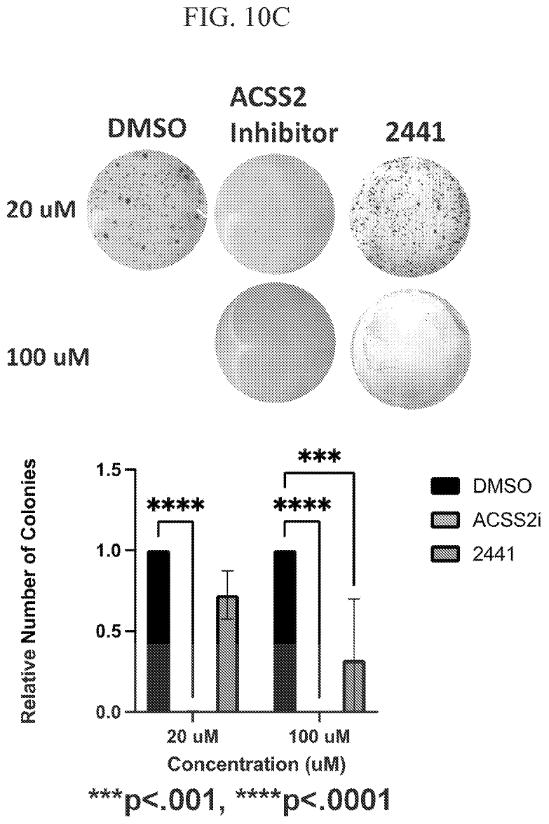

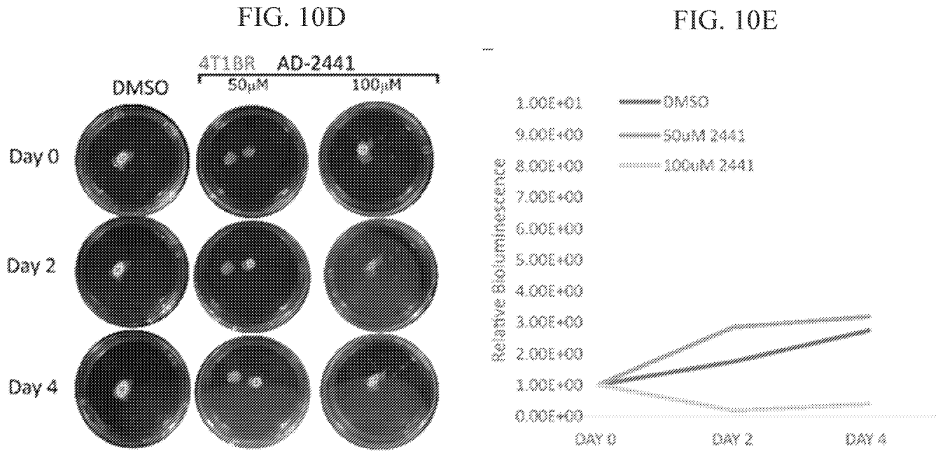

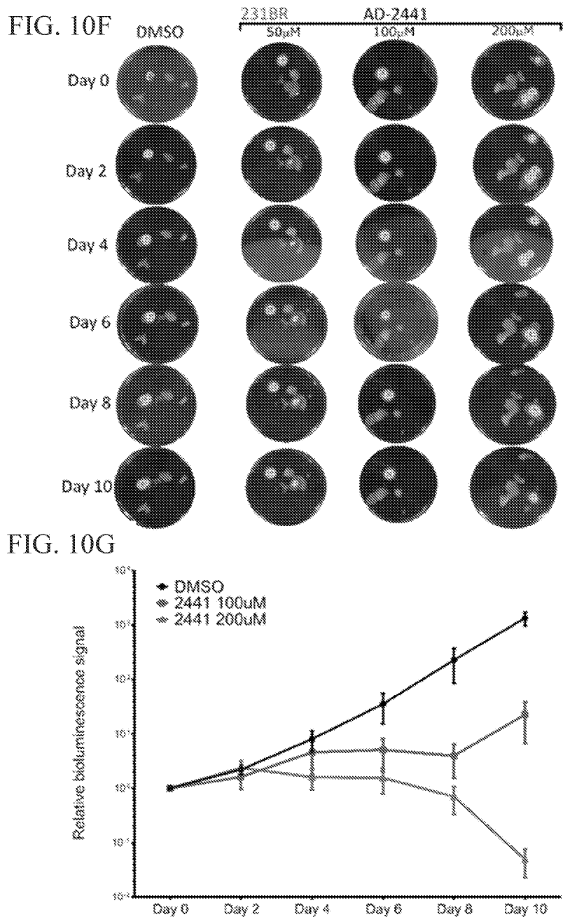

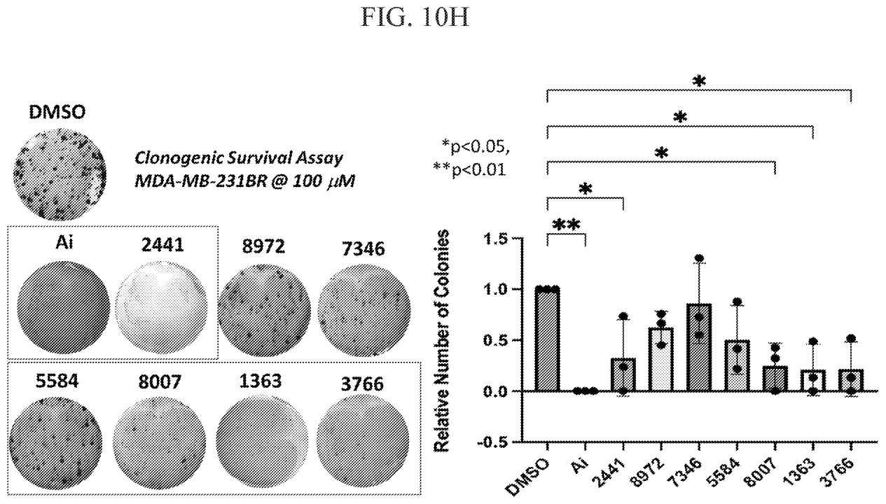

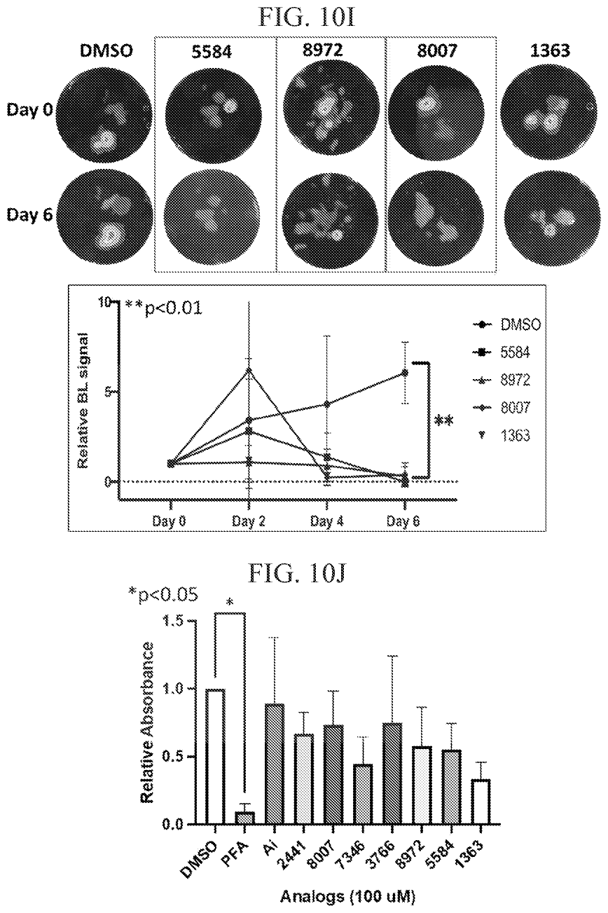

Triple-negative brain trophic cells MDA-MB-231BR (Center for Cancer Research, National Cancer Institute) were cultured with 10% fetal bovine serum (FBS), 5% 10,000 Units/mL Penicillin-10,000 μg/mL Streptomycin, and 5% 200 mM L-Glutamine. Using the breast cancer brain metastatic MDA-MB-231-BR cells in a crystal violet assay, AD-2441 was shown to significantly inhibit (n=4, p<0.05) cancer cell proliferation (FIG. 10A, FIG. 10B). For crystal violet staining, 5×10⁴ cells were plated in to a 6-well plate and subjected to the treatments as described in the individual figures and then stained with 0.5% crystal violet prepared in a 1:1 methanol-water solution followed by dH2O washes. This finding was confirmed this using a clonogenicity assay to determine reproductive cell death after treatment with AD-2441 (FIG. 10C). Moreover, using an ex vivo BCBM mouse brain slice model, AD-2441 was shown to suppress BCBM growth in two breast cancer brain metastatic cell lines, 4T1-BR (FIG. 10D, FIG. 10E) and MDA-MB-231-BR (FIG. 10F, FIG. 10G).

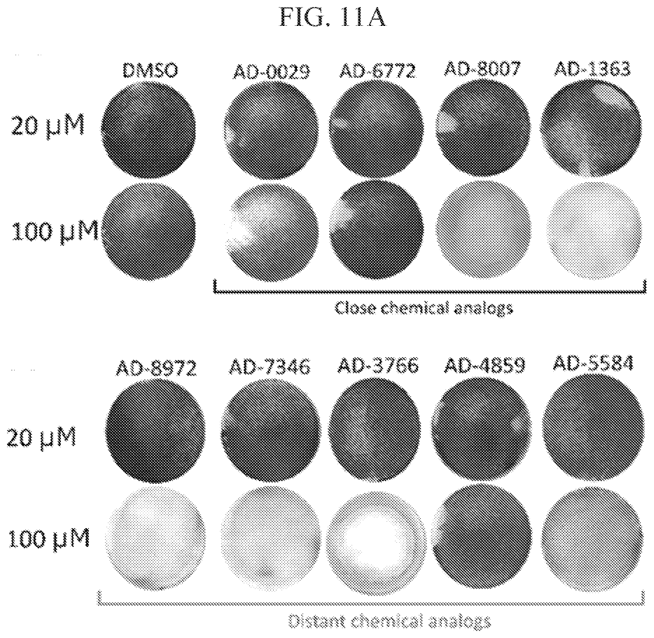

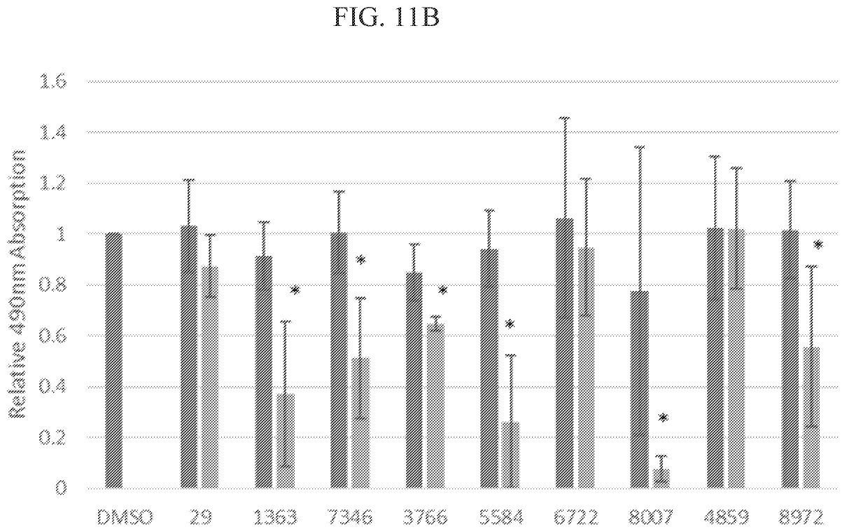

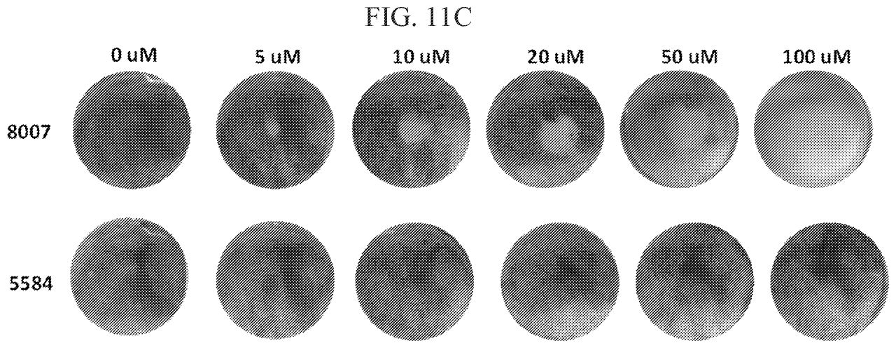

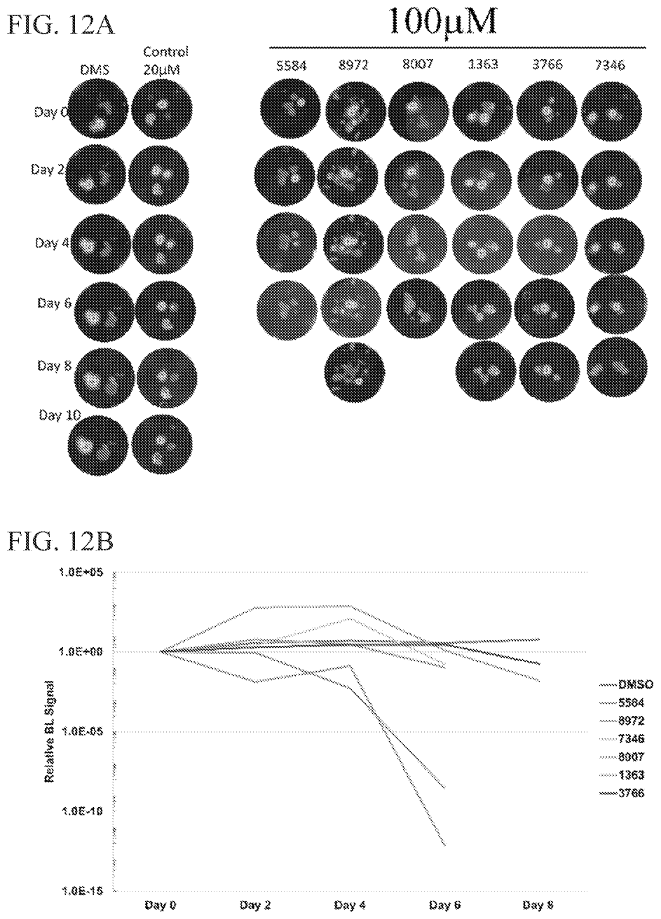

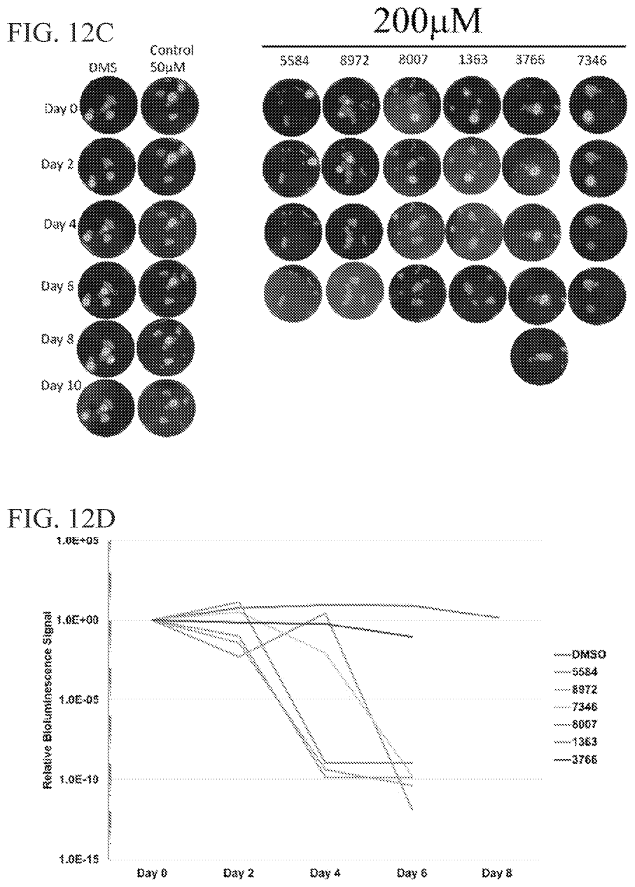

The nine close (AD-0029, AD-6772, AD-8007, AD-1363) and distant (AD-8972, AD-7346, AD-3766, AD-4859, AD-5584) chemical analogs of AD-2441 were assayed. All 9 analogs engaged hACSS-2 directly (FIG. 8A, FIG. 8B, FIG. 9, Table 1, Table 2) with improved low μM affinities as compared to the parental AD-2441, and in preliminary experiments, all analogs inhibited cancer cell proliferation (FIG. 11A, FIG. 11B, FIG. 11C) using a crystal violet assay and MDA-MB-231-BR tumor cells. Using an ex vivo BCBM mouse brain slice model, AD-2441 analogs were shown to suppress MDA-MB-231-BR tumor growth (FIG. 12A, FIG. 12B, FIG. 12C, FIG. 12D).

BODIPY Staining of Cells. Cells were treated with 6 μM BODIPY in PBS for 15 min, washed 2× with 1×PBS, fixed using 4% paraformaldehyde for 30 min at RT in the dark and washed in 1×PBS prior to mounting and imaging on EVOS FL (Life Technologies) using Texas Red filter.

For crystal violet staining, 5×10⁴ cells were plated and subjected to the treatments as described in the individual figures and then stained with 0.5% crystal violet prepared in a 1:1 methanol-water solution followed by PBS washes. For quantification, crystal violet was dissolved in 1% deoxycholate and absorbance was measured at 490 nm.

To assess clonogenic survival, cells were cultured and detached in their respective growth media, then counted using a hemocytometer. 1,000 cells were plated in triplicate in 6-well plates in their respective growth media and allowed to form colonies. Cells were grown for 14 days. Upon conclusion of growth period, colonies were stained with crystal violet. Colony numbers were quantified based on number of colonies with >40 cells. For clonogenic survival based on drug exposure, 100,000 cells were plated in a 6-well plate, then 24 hours later treated with noted concentration of drug for 48 hours. After 48 hours, cells in each concentration condition were lifted, counted, and plated per the described clonogenic survival protocol and allowed to grow for noted length of time in noted growth media. Upon conclusion of growth period, colonies were stained with crystal violet. Colony numbers were quantified based on number of colonies with >40 cells.

Ex vivo brain slice model. Organotypic hippocampal cultures were prepared as described previously (PMID: 34633392). Briefly, adult mice (6-8 week) or mice after 12 days following intracranial injection were decapitated and their brains rapidly removed into ice-cold (4° C.) sucrose-aCSF composed of the following (in mM): 280 sucrose, 5 KCl, 2 MgCl2, 1 CaCl2, 20 glucose, 10 HEPES, 5 Na+-ascorbate, 3 thiourea, 2 Na+-pyruvate; pH=7.3. Brains were blocked with a sharp scalpel and sliced into 250 μm sections using a McIlwain-type tissue chopper (Vibrotome inc). Four to six slices were placed onto each 0.4 μm Millicell tissue culture insert (Millipore) in six-well plates, 1 ml of medium containing the following: Neurobasal medium A (Gibco), 2% Gem21-Neuroplex supplement (Gemini), 1% N2 supplement (Gibco), 1% glutamine (Invitrogen), 0.5% glucose, 10 U/ml penicillin, and 100 μg/ml streptomycin (Invitrogen), placed underneath each insert. One-third to one half of the media was changed every 2 d. Tumor growth was monitored via bioluminescence imaging on the IVIS 200 system (Perkin Elmer) and results analyzed using Living Image software (Caliper Life Sciences, Waltham, MA, USA). For MTS assay, individual brain slices were transferred to a 96-well plate and subjected to Promega CellTiter 96® Aqueous One Solution (Cat: G3582) mixed in a 1:5 ratio with culture media and treated as previously described. Tissues were incubated at 37° C., 5% CO2 for 4 hours and absorbance at 490 nm was measured with Tecan Spark Microplate reader.

### Example 4

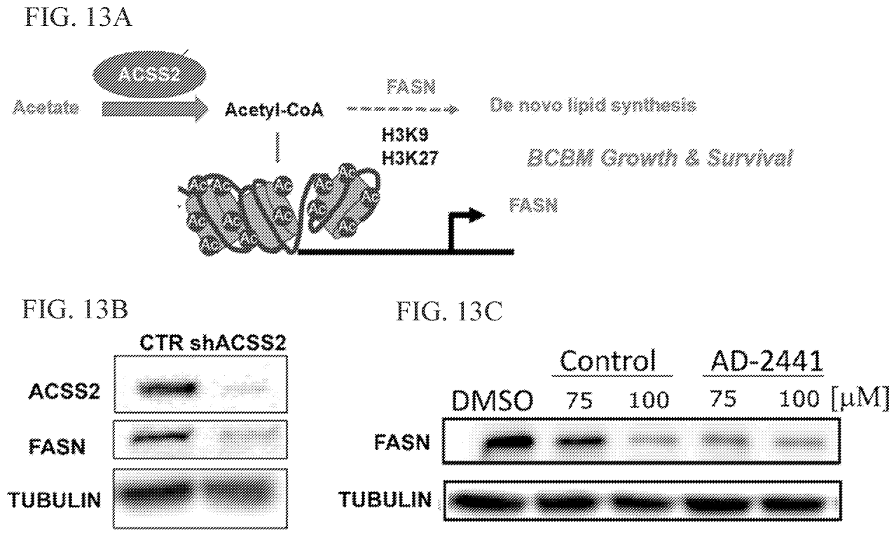

ACSS-2 provides Acetyl-CoA for numerous metabolic processes vital for cancer proliferation. The human fatty acid synthase (FASN) is essential for BCBM growth and survival (FIG. 13A), and hACSS-2 does regulate FASN protein expression levels BCBM cells (FIG. 13B). AD-2441 downregulates FASN expression levels and possibly reduces de novo lipid synthesis essential for BCBM tumor growth (FIG. 13C).

### Example 5

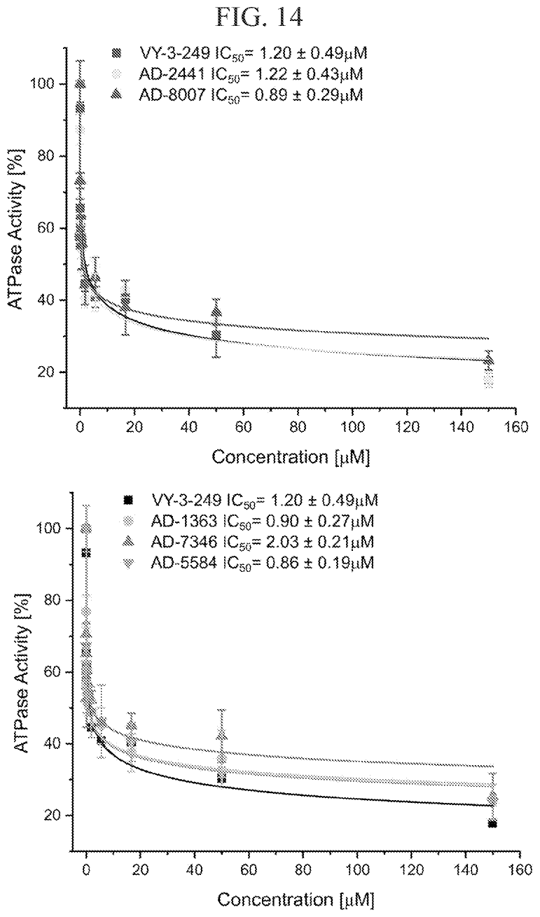

ACSS2 enzyme activity was measured using the TranScreener TRF AMP/GMP assay (Bellbrook Labs) (FIG. 14). In house recombinant hACSS2 was used. The assay was performed in white, opaque, low volume 96-well plates. Test compounds were diluted in 100% DMSO and used at the indicated final concertation. hACSS2 (3 nmol/L) in assay buffer (30 mmol/L HEPES, pH 7.4, 140 mmol/L NaCl, 2 mmol/L MgCl2, 5 mmol/L sodium acetate, 2 mmol/L DTT, 0.005% Brij35). Three microliters of substrate mix containing 100 mmol/L ATP and 10 mmol/L CoA was added to followed by a 120-minute incubation. Final substrate concentrations were 5 mmol/L acetate, 50 mmol/L ATP, and 5 mmol/L CoA in the acetate reaction. After incubation, 3 mL of terbium conjugated AMP antibody and AMP tracer was added to according to the methods described by BellBrook Labs. After 30 minutes, the Fluorescent polarization signal was measured using an Teacon Spark Plate reader. Data were normalized to percent activity, where 100% inhibition equals the counts obtained in absence of ACSS2, and 0% inhibition equals the counts obtained in the complete reaction including a DMSO control.

### Example 6

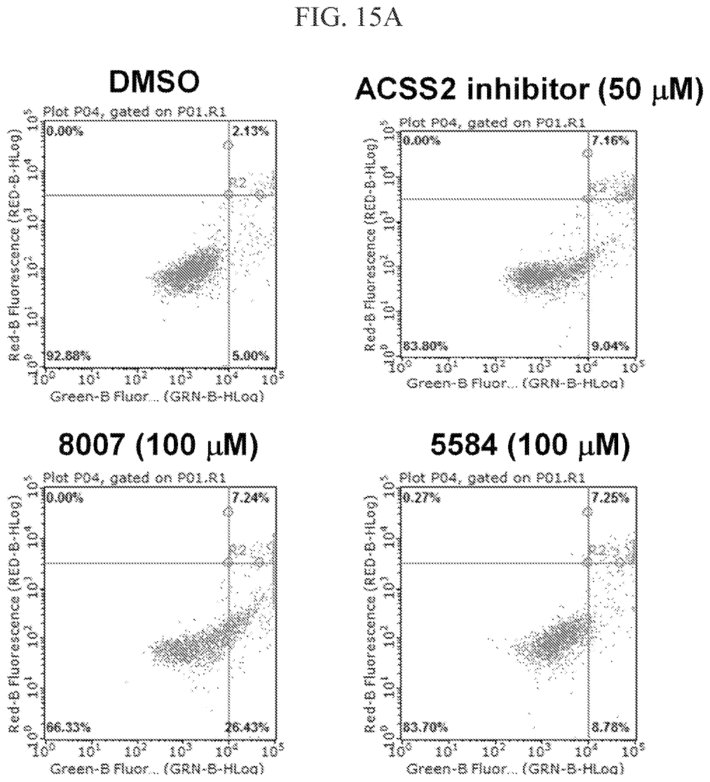

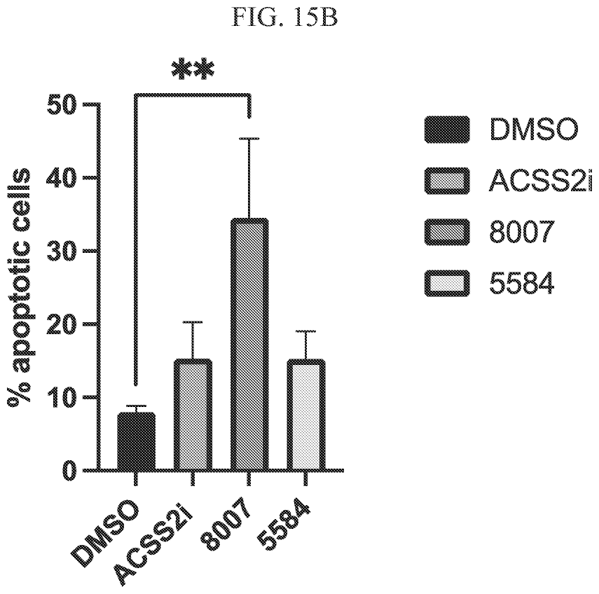

ACSS2 inhibitors on the induction of apoptosis in BCBM cells. Cells were treated with ACSS2 inhibitors for 24 hours, washed with PBS, trypsinized (0.05% Trypsin), counted, 1×10⁶ cells were resuspended in PBS, pelleted, and the supernatant was removed. Cells were resuspended 100 μL of in 1× binding buffer in a 1.7 mL Eppendorf tube with 5 μL Annexin and 5 μL Propidine Iodine (PI) at room temp in the dark for 30 minutes. Then 400 μL of 1× binding buffer was added to the tubes, then read on Guava easyCyte flow cytometer. All data were collected and analyzed using a Guava EasyCyte Plus system and CytoSoft (version 5.3) software (Millipore). Data are gated and expressed relative to the appropriate unstained and single stained controls. FITC Annexin V Apoptosis Detection Kit containing binding buffer, Annexin V and PI were purchased from BD Biosciences (Cat: 556547). The effect of ACSS2 inhibitors on the induction of apoptosis in BCBM cells is shown in FIG. 15A and FIG. 15B.

### Example 7

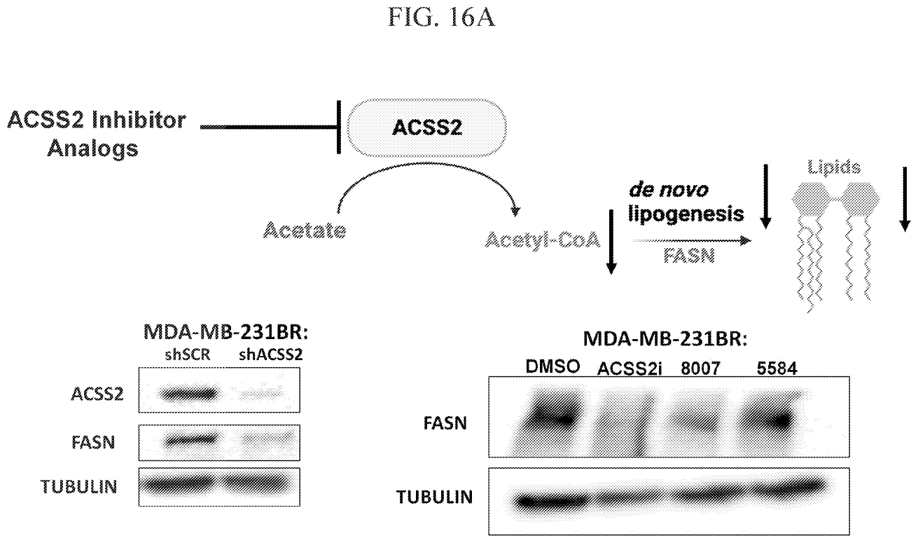

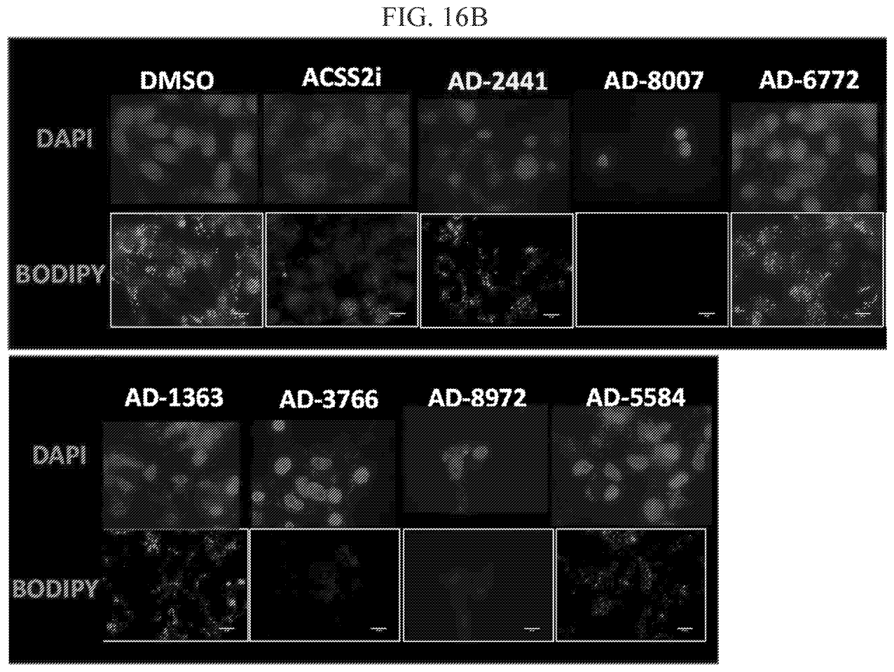

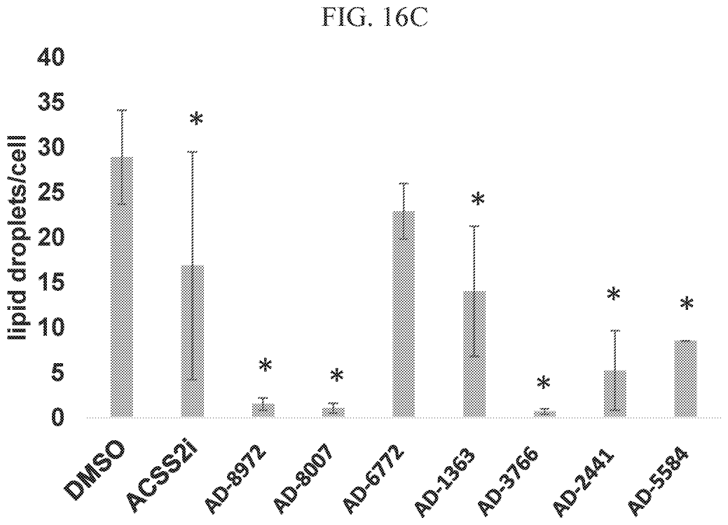

ACSS2 inhibitors on levels of lipid droplet storage. Briefly, cell lysates from 1-5×10⁶ cells were prepared in radioimmune precipitation assay buffer (150 mM NaCl, 1% NP40, 0.5% DOC, 50 mM Tris HCL at pH 8, 0.1% SDS, 10% glycerol, 5 mM EDTA, 20 mM NaF, and 1 mM Na3 VO4) supplemented with 1 μg/ml each of pepstatin, leupeptin, aprotinin, and 200 μg/ml PMSF. Lysates were cleared by centrifugation at 16,000×g for 15 minutes at 4° C. and analyzed by SDS-PAGE and autoradiography with chemiluminescence. Proteins were analyzed by immunoblotting using primary antibodies against Fatty Acid Synthase (FASN) (Cell Signaling) and beta-Tubulin (Cell Signaling) (FIG. 16A).

For BODIPY Staining of cells, cells were treated with 6 μM BODIPY in PBS for 15 min, washed 2× with 1×PBS, fixed using 4% paraformaldehyde for 30 min at RT in the dark and washed in 1×PBS prior to mounting and imaging on EVOS FL (Life technologies) using Texas Red filter. The effect of ACSS2 inhibitors on levels of lipid droplet storage is shown in FIG. 16A, FIG. 16B, and FIG. 16C.

### Example 8

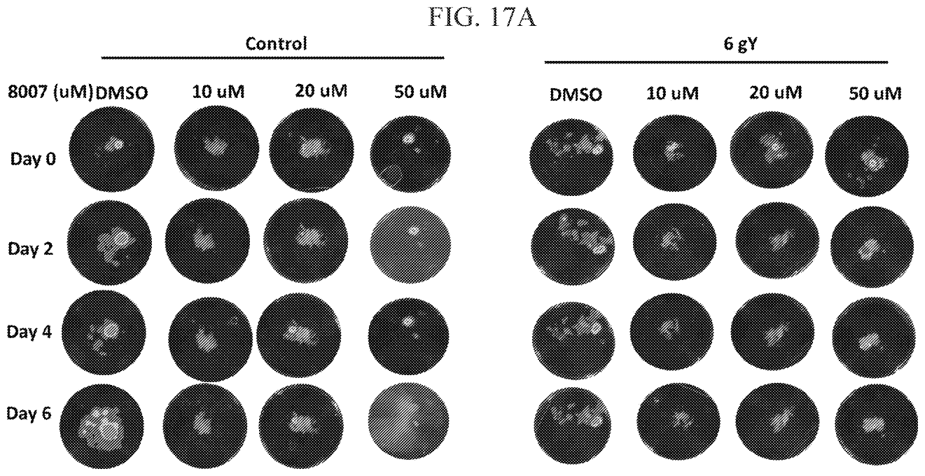

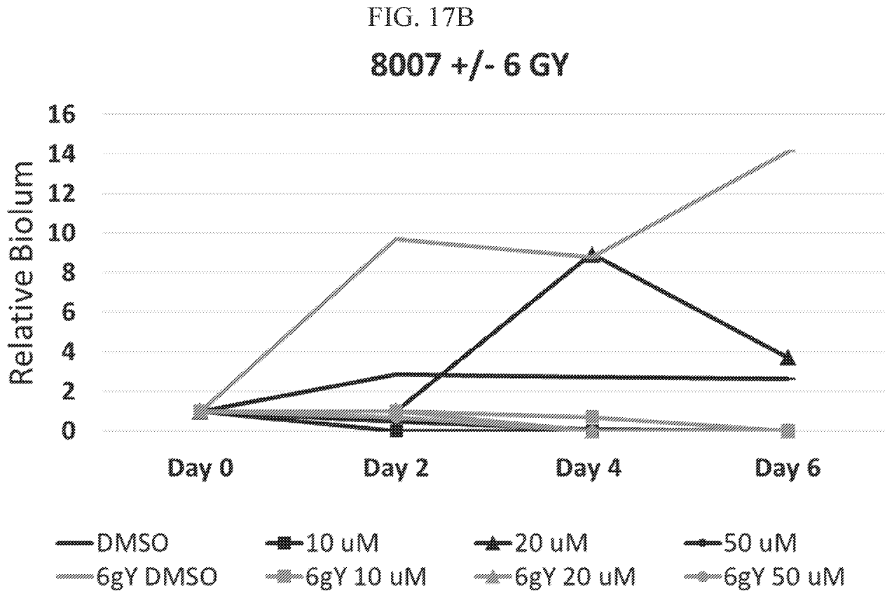

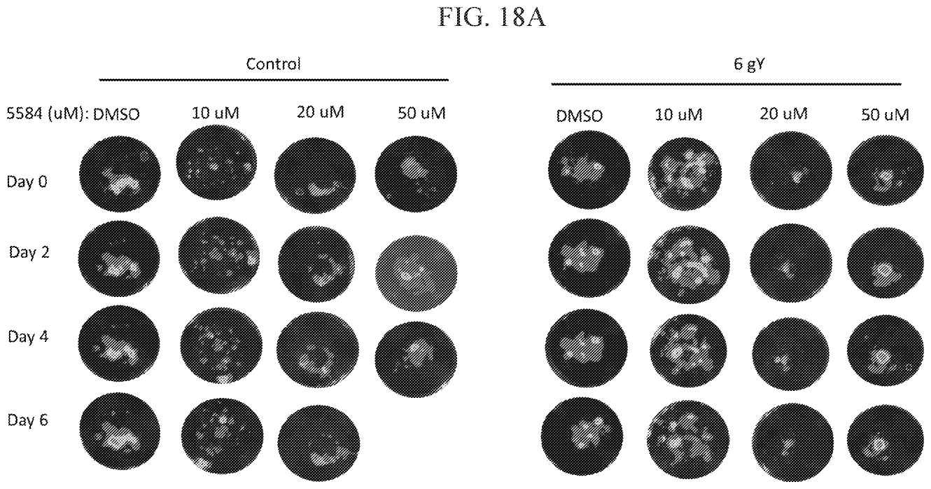

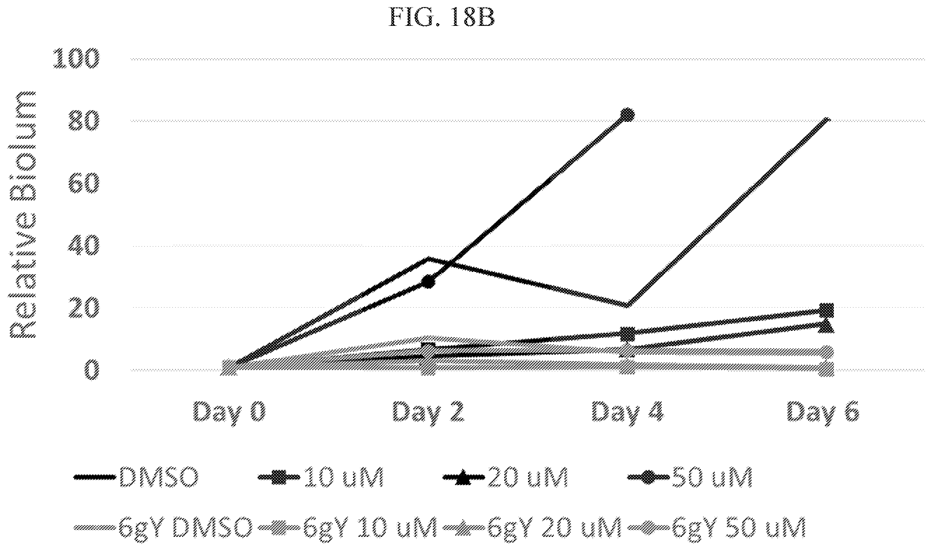

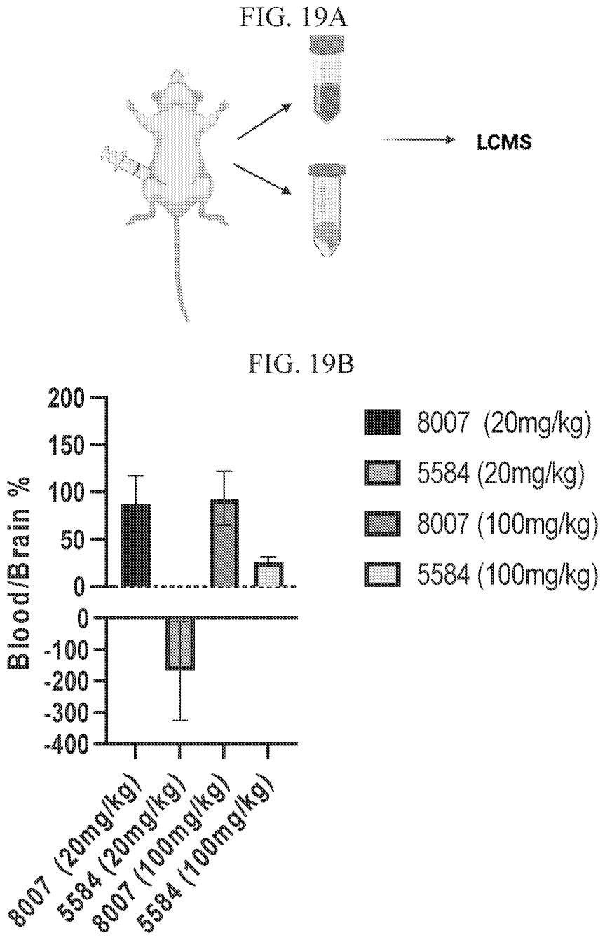

The effects of the ACSS2 Inhibitors on luciferase tagged MDA-MB-231BR tumor-brain slices in combination with sample irradiation was assessed. The ex vivo mouse brain slice model was performed as described herein. The results of combination therapy with the indicated ACSS2 inhibitor and irradiation are shown in FIG. 17A, FIG. 17B, 18A, and FIG. 18B.

### Example 9

Metabolic Stability and Plasma Stability. Test compound Working solution: 5 μL of compound stock solution (10 mM in dimethyl sulfoxide (DMSO)) were diluted with 495 μL of DMSO (Working solution concentration: 100 μM, 100% DMSO). Propantheline bromide Working solution: 5 μL of Propantheline bromide stock solution (10 mM in H2O) were diluted with 495 μL of H2O (Working solution concentration: 100 μM, 100% H2O)

Human plasma from 3 males and 3 females was obtained. Heparin was used as the anticoagulant. The pooled frozen plasma was thawed in a water bath at 37° C. prior to experiment. Plasma was centrifuged at 4000 rpm for 5 min and the clots were removed if any. Using an Apricot automation workstation, 98 μL/well of blank plasma were added to all 96-well reaction plates. (Blank, T0, T10, T30, T60 and T120). The appointed incubation time points are T0 (0 min), Tn (n=0, 10, 30, 60, 120 min). An Apricot automation workstation was used to add 2 μL/well of working solution (100 μM) to all reaction plates except Blank. (T0, T10, T30, T60 and T120). All reaction plates containing mixtures of compound and plasma were incubated at 37° C. in water bath. The reaction plates were incubated at 37° C., and timer was started. At the end of incubation, 500 μL of stop solution was added and mixed (200 ng/mL tolbutamide and 200 ng/ml labetalol in ACN) to precipitate protein. Each plate was sealed and shaken for 20 minutes. After shaking, each plate was centrifuged at 4000 rpm and 4° C. for 20 minutes. After centrifugation, an Apricot automation workstation was used to transfer 150 μL of supernatant from each reaction plate to its corresponding bioanaylsis plate. Each bioanalysis plate was sealed and shaken for 10 minutes prior to LC-MS/MS analysis. The % remaining of test compound after incubation in plasma was calculated using following equation: Remaining=100×(PAR at appointed incubation time/PAR at T0 time) where PAR is the peak area ratio of analyte versus internal standard (IS).

TABLE 3
Experimental Validation (Metabolic Stability, Plasma Stability)

| | | | | | | | Human |
|---|---|---|---|---|---|---|---|
| | | | | | | | Plasma |
| | Human Liver Microsome Stability | | | | | | Stability |
| | | T_{1/2} | | | Remaining(T = | Remaining(NCF = | T_{1/2} |
| Compound ID | R² | (min) | CLint(mic)(μL/min/mg) | CLint(liver)(mL/min/kg) | 60 min) | 60 min) | (min) |
| AD-8007 | 0.9824 | >145 | <9.6 | <8.6 | 0.77227 | 0.95633 | >289.1 |
| AD-3766 | 1 | 0.914 | 1516.099 | 1364.4891 | 0.0005 | 0.96386 | >289.1 |
| AD-5584 | 0.983 | 19.565 | 70.842 | 63.7578 | 0.12368 | 0.83493 | >289.1 |
| Testosterone | 0.9884 | 15.519 | 89.309 | 80.3781 | 0.06959 | 0.89758 | |
| Diclofenac | 0.9939 | 5.293 | 261.875 | 235.6875 | 0.00044 | 0.87629 | |
| Propafenone | 0.9182 | 7.919 | 175.015 | 157.5135 | 0.00396 | 0.93177 | |
| Propantheline | | | | | | | 18.6 |
| Bromide | | | | | | | |

### Example 10

MDCKI MDR1 cell permeability assessment was performed to mimic the blood brain barrier. MDR1-MDCK I cells were seeded onto polyethylene membranes (PET) in 96-well Corning insert systems at 2.5×10⁵ cells/mL until to 4-7 days for confluent cell monolayer formation. The transport buffer in the study was HBSS with 10.0 mM HEPES at pH 7.40+0.05. Test compounds were tested at 2.00 μM bi-directionally in duplicate. Digoxin was tested at 10.0 μM bi-directionally in duplicate, while nadolol and metoprolol were tested at 2.00 μM in A to B direction in duplicate. Final DMSO concentration was adjusted to less than 1%. The plate was incubated for 1.5 hours in CO2 incubator at 37±1° C., with 5% CO2 at saturated humidity without shaking. All samples after mixed with acetonitrile containing internal standard were centrifuged at 3220×g for 10 min. For all samples, 150 μL supernatant solution was diluted with 150 μL ultra-pure water for LC-MS/MS analysis. In addition, the efflux ratio of each compound was also determined. Test and reference compounds were quantified by LC-MS/MS analysis based on the peak area ratio of analyte/IS. After transport assay, Lucifer yellow rejection assay are applied to determine the cell monolayer integrity. Buffers are removed from both apical and basolateral chambers, followed by the addition of 75 μL of 100 μM lucifer yellow in transport buffer and 250 μL transport buffer in apical and basolateral chambers, respectively. The plate is incubated for 30 minutes at 37° C. with 5% CO2 and 95% relative humidity without shaking. After 30 minutes incubation, 20 μL of lucifer yellow samples are taken from the apical sides, followed by the addition of 60 μL of Transport Buffer. And then 80 μL of lucifer yellow samples are taken from the basolateral sides. The relative fluorescence unit (RFU) of lucifer yellow is measured at 425/528 nm (excitation/emission) with a microplate reader.

The apparent permeability coefficient Papp (cm/s) was calculated using the equation: Papp=(dCr/dt)×Vr/(A×C0) where dCr/dt is the cumulative concentration of compound in the receiver chamber as a function of time (μM/s); Vr is the solution volume in the receiver chamber (0.075 mL on the apical side, 0.25 mL on the basolateral side); A is the surface area for the transport, i.e. 0.0804 cm² for the area of the monolayer; CO is the initial concentration in the donor chamber (μM). The efflux ratio was calculated using the equation: Efflux Ratio=Papp (BA)/Papp (AB) Percent recovery was calculated using the equation: % Solution Recovery=100×[(Vr×Cr)+(Vd×Cd)]/(Vd×CO) Where Vd is the volume in the donor chambers (0.075 mL on the apical side, 0.25 mL on the basolateral side); Cd and Cr are the final concentrations of transport compound in donor and receiver chambers, respectively. Percent of lucifer yellow in basolateral well is calculated using the equation: Where RFUApical and RFUBasolateral are the relative fluorescence unit values of lucifer yellow in the apical and basolateral wells, respectively; VApical and VBasolateral are the volume of apical and basolateral wells (0.075 mL and 0.25 mL), respectively. The % Lucifer Yellow should be less than 1.0.

TABLE 4
Bi-directional Permeability Across MDR1-MDCK I Cell Monolayer Mimicking the Blood Brain Barrier

| | Mean Pₐₚₚ (10 [symbol] cm/s) | | Efflux | Mean % Solution Recovery | | |
|---|---|---|---|---|---|---|
| Compound ID | A to B | B to A | Ratio | A to B | B to A | Note |
| Nadolol | 0.257 | ND | ND | 99.1 | ND | Low permeability marker |
| Metoprolol | 12.9 | ND | ND | 94.2 | ND | High permeability marker |
| Digoxin | 0.0595 | 7.74 | 130 | 84.3 | 100.4 | P-gp substrate |
| AD-8007 | 2.81 | 25.2 | 8.96 | 81.3 | 97.0 | AD 8007 has a low efflux ratio |
| | | | | | | and potential to cross the BBB |
| AD-5584 | 2.11 | 44.4 | 21.0 | 94.9 | 111.6 | — |

ND means not determined [symbol]
indicates data missing or illegible when filed

### Example 11

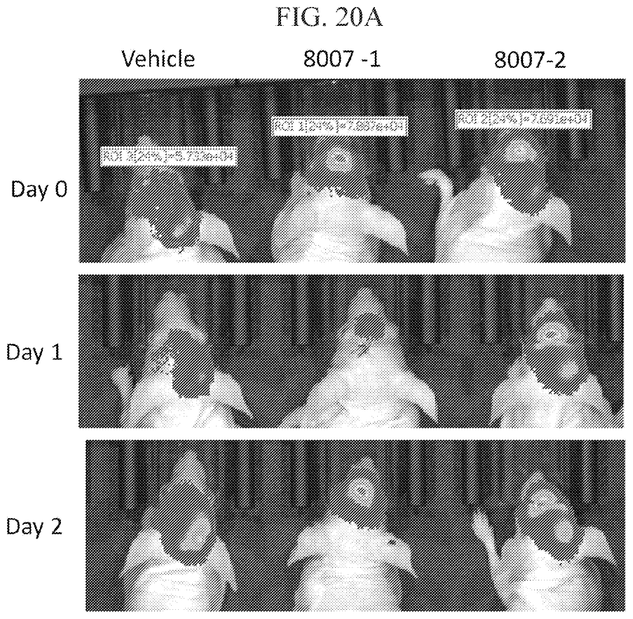

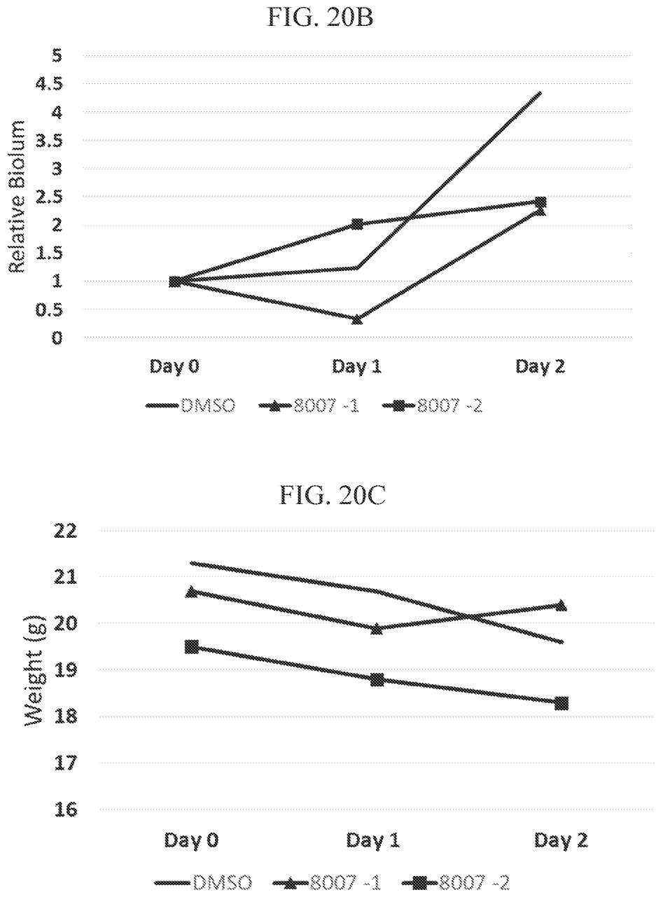

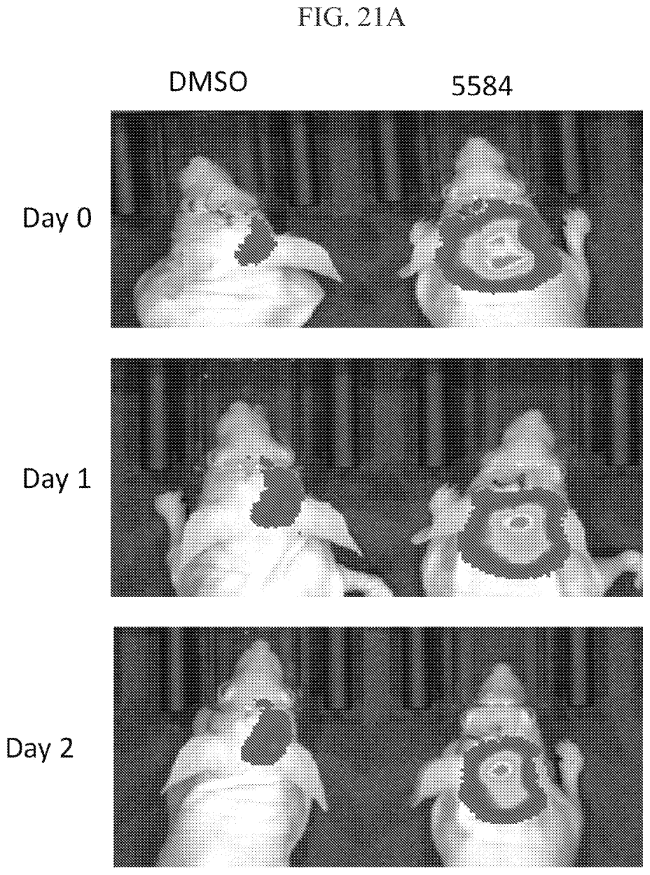

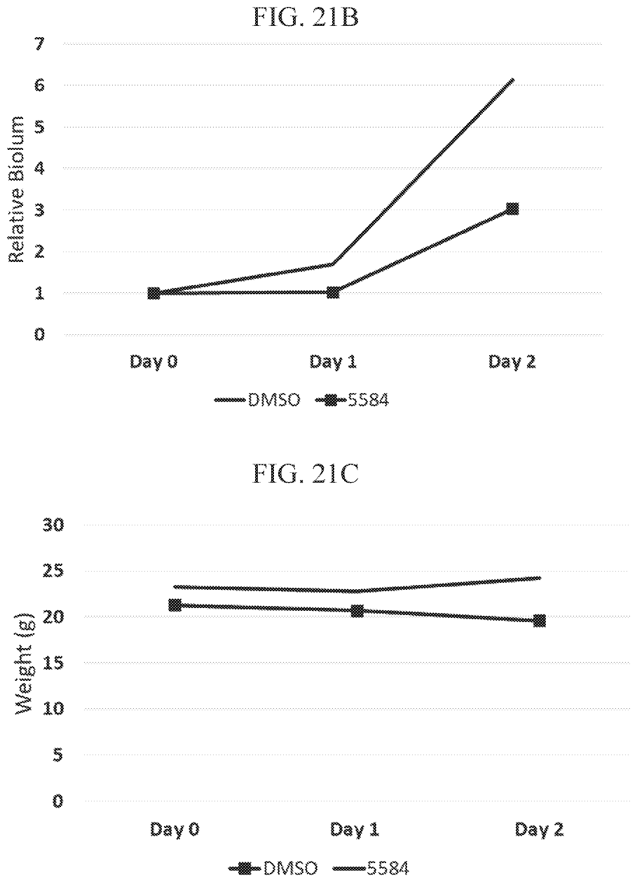

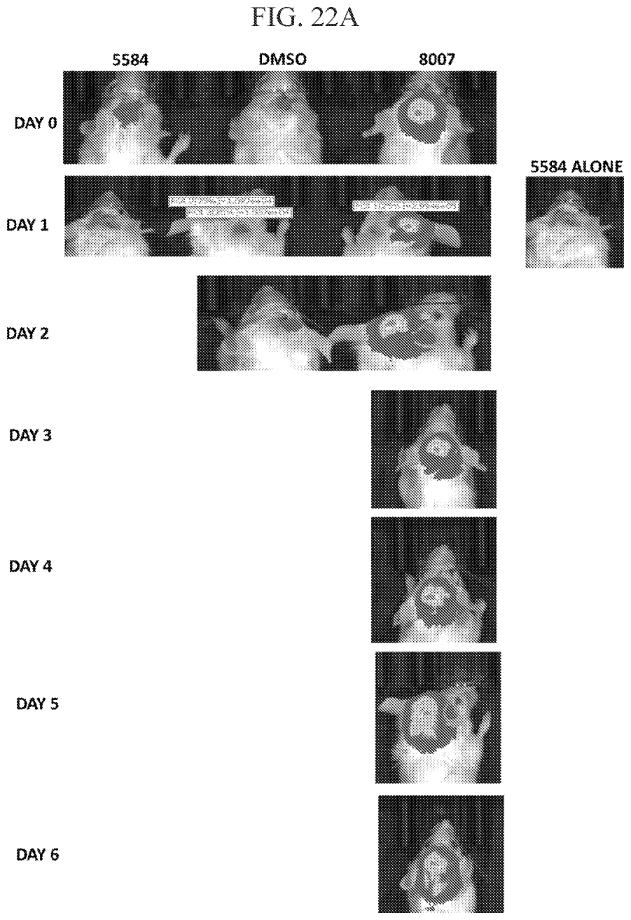

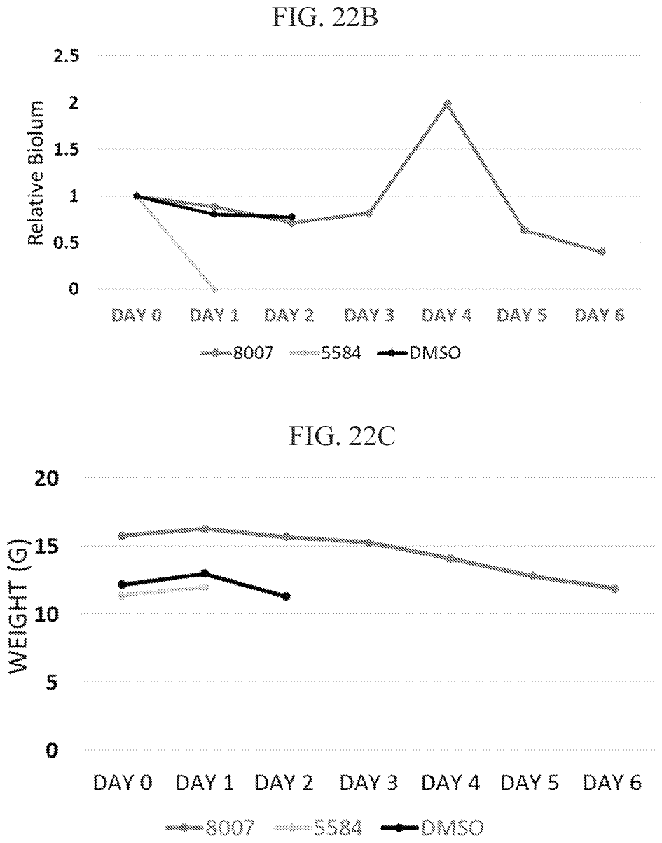

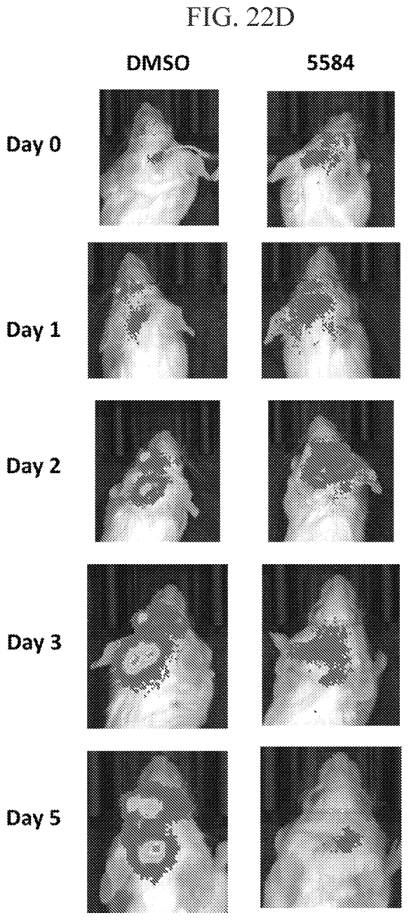

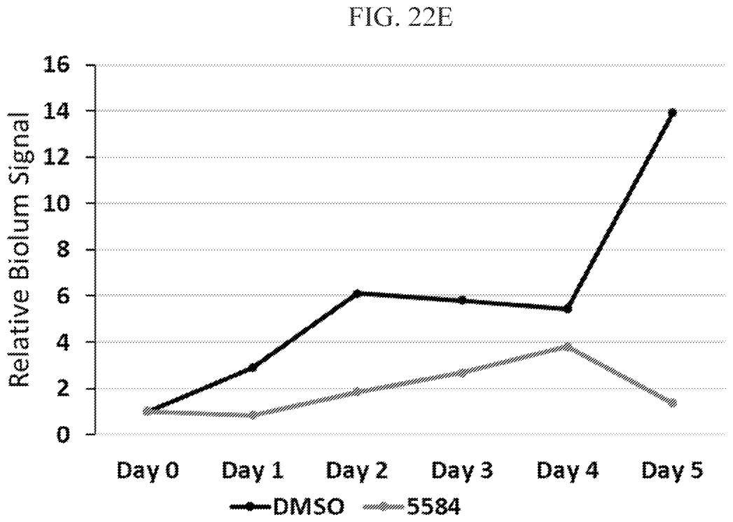

In vivo efficacy of the ACSS2 inhibitors. Nu/Nu athymic 6-8 week old mice from Charles River Laboratories (Wilmington, MA, USA) were immobilized using the Just for Mice TM Stereotaxic Frame (Harvard Apparatus, Holliston, MA, USA) and injected intracranially with 5 μL of a 20,000 cells/μL solution of MDA-MB-231BR cells stably expressing luciferase. Mice were injected intraperitoneally with 200 μl of Dluciferin solution (30 mg/ml; Caliper Life Sciences, Hopkinton, MA) and results analyzed using Living Image software (Caliper Life Sciences, Waltham, MA, USA). For intracranial injections, mice were euthanized 3 weeks after injection. The in vivo efficacy of the ACSS2 inhibitors are shown in FIG. 20A, FIG. 20B, FIG. 20C, FIG. 21A, FIG. 21B, FIG. 21C, FIG. 22A, FIG. 22B, FIG. 22C. FIG. 22D. FIG. and 22E.

Those skilled in the art will appreciate that numerous changes and modifications can be made to the preferred embodiments of the methods disclosed and that such changes and modifications can be made without departing from the spirit of the methods. It is, therefore, intended that the appended claims cover all such equivalent variations as fall within the true spirit and scope of the methods.

The disclosures of each patent, patent application, and publication cited or described in this document are hereby incorporated herein by reference, in its entirety.

### EMBODIMENTS

1. A method of treating a cancerous disease in a subject, the method comprising: administering to the subject a therapeutically effective amount of any one or more of the following compounds:

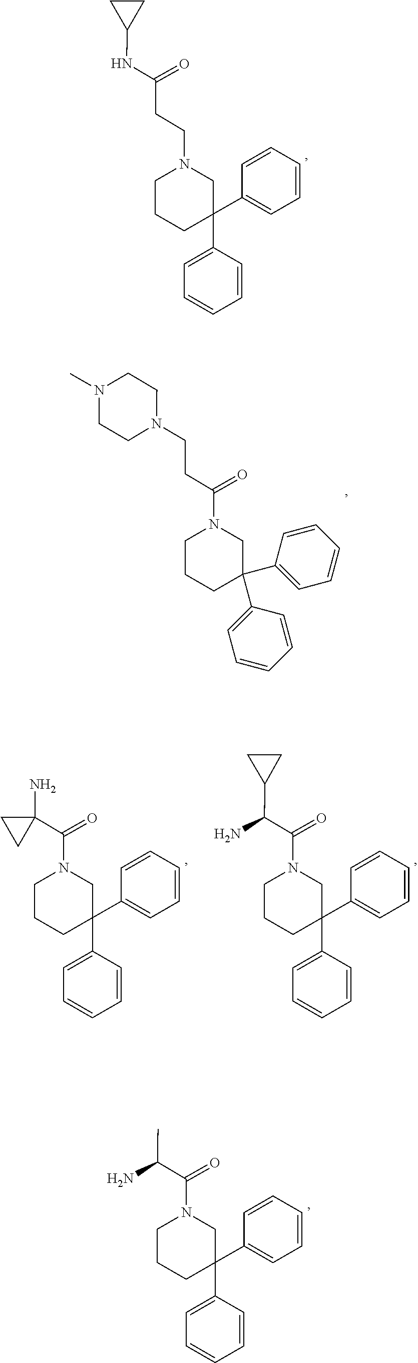 [SMILES: O=C(CCN1CCCC(c2ccccc2)(c2ccccc2)C1)NC1CC1 ; CN1CCN(CCC(=O)N2CCCC(c3ccccc3)(c3ccccc3)C2)CC1 ; NC1(C(=O)N2CCCC(c3ccccc3)(c3ccccc3)C2)CC1 ; N[C@H](C(=O)N1CCCC(c2ccccc2)(c2ccccc2)C1)C1CC1 ; C[C@H](N)C(=O)N1CCCC(c2ccccc2)(c2ccccc2)C1]

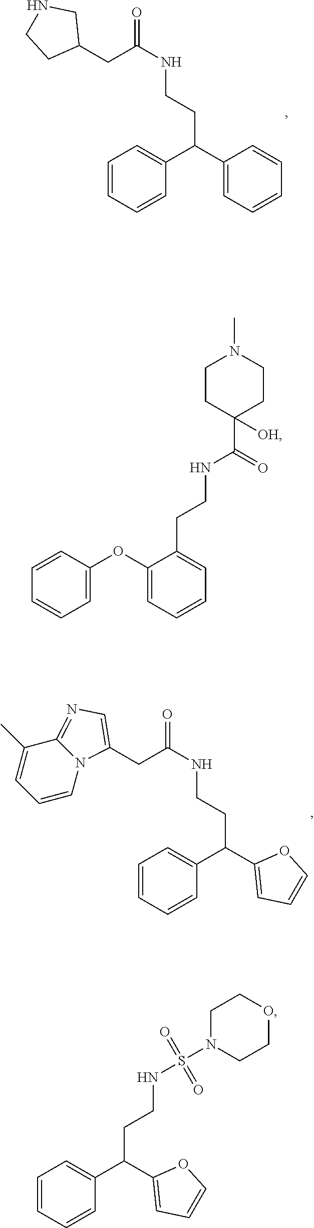 [SMILES: O=C(CC1CCNC1)NCCC(c1ccccc1)c1ccccc1 ; CN1CCC(O)(C(=O)NCCc2ccccc2Oc2ccccc2)CC1 ; Cc1cccn2c(CC(=O)NCCC(c3ccccc3)c3ccco3)cnc12 ; O=S(=O)(NCCC(c1ccccc1)c1ccco1)N1CCOCC1]

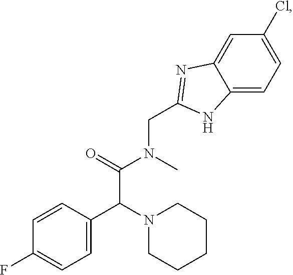 [SMILES: CN(Cc1nc2cc(Cl)ccc2[nH]1)C(=O)C(c1ccc(F)cc1)N1CCCCC1]

or a pharmaceutically acceptable salt thereof.
2. The method of embodiment 1, wherein the cancerous disease is a brain cancer.
3. The method of embodiment 2, wherein the brain cancer is a brain metastasis.
4. The method of embodiment 3, wherein the brain metastasis is a breast cancer brain metastatic tumor.
5. The method of any one of the preceding embodiments, wherein the compound crosses the blood-brain barrier.
6. The method of embodiment 1, wherein the cancerous disease is breast cancer.
7. The method of any one of the preceding embodiments, wherein the compound inhibits and/or inactivates acyl-coenzyme A synthetase short-family member 2 (ACSS-2).
8. The method of any one of the preceding embodiments, wherein growth of the cancerous disease is inhibited.
9. The method of any one of the preceding embodiments, further comprising administering radiation therapy to the subject.
10. The method of embodiment 9, wherein the radiation therapy is directed to a tumor.
11. A method of inhibiting and/or inactivating acyl-coenzyme A synthetase short-family member 2 (ACSS-2), the method comprising contacting the ACSS-2 with of any one or more of the following compounds:

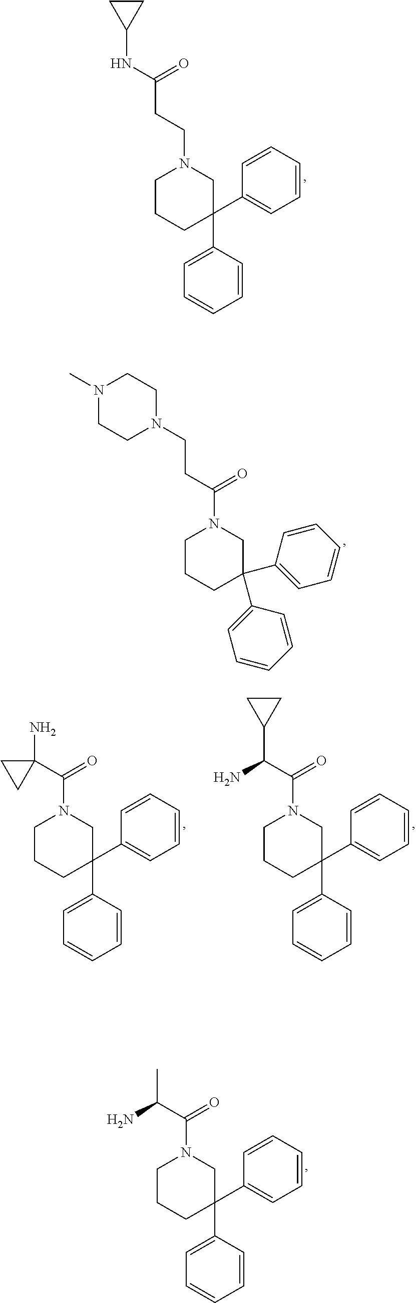

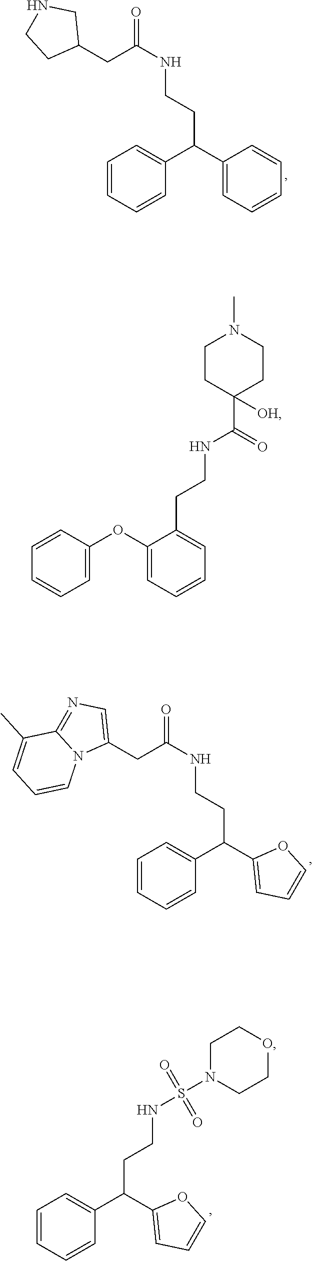 [SMILES: O=C(CC1CCNC1)NCCC(c1ccccc1)c1ccccc1 ; CN1CCC(O)(C(=O)NCCc2ccccc2Oc2ccccc2)CC1 ; Cc1cccn2c(CC(=O)NCCC(c3ccccc3)c3ccco3)cnc12 ; O=S(=O)(NCCC(c1ccccc1)c1ccco1)N1CCOCC1]

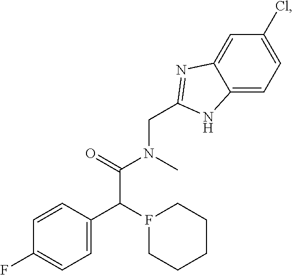

or a pharmaceutically acceptable salt thereof.

1. A method of treating a cancerous disease in a subject, the method comprising: administering to the subject a therapeutically effective amount of any one or more of the following compounds: 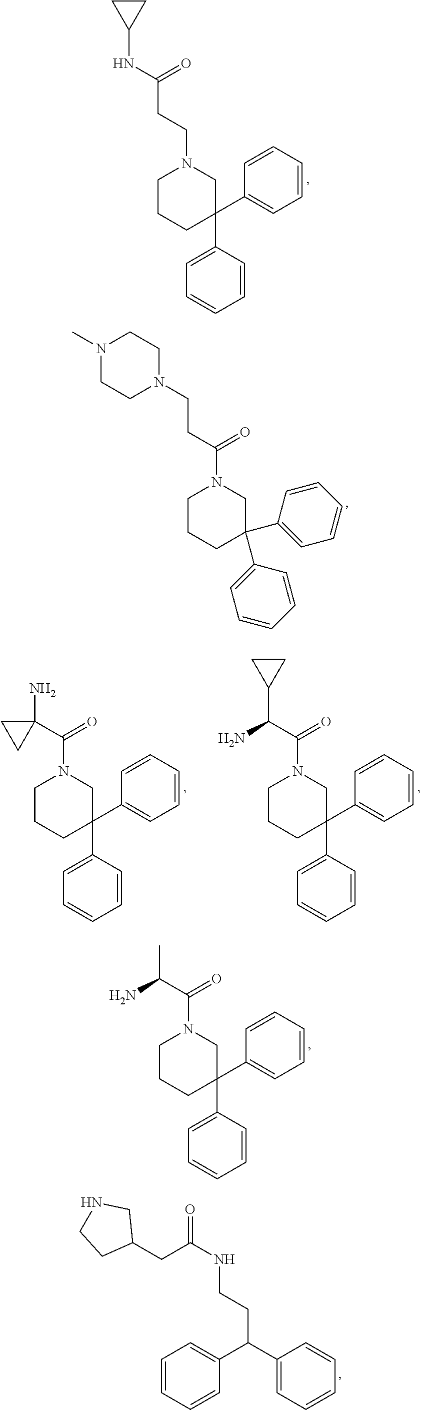 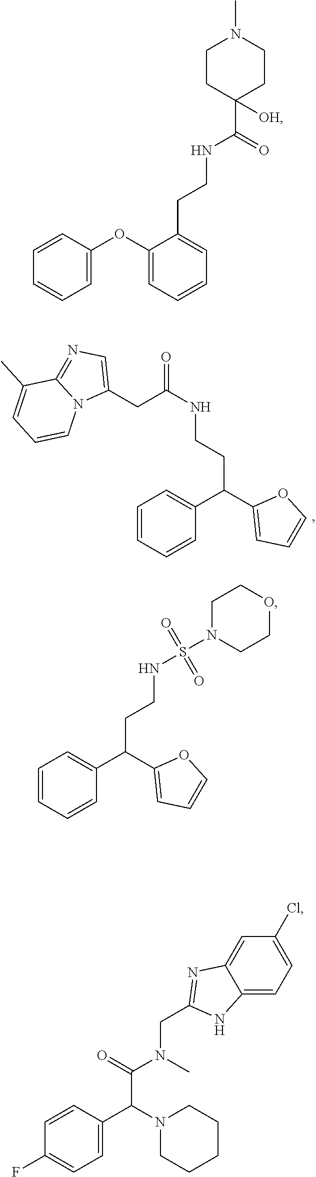 [SMILES: CN1CCC(O)(C(=O)NCCc2ccccc2Oc2ccccc2)CC1 ; Cc1cccn2c(CC(=O)NCCC(c3ccccc3)c3ccco3)cnc12 ; O=S(=O)(NCCC(c1ccccc1)c1ccco1)N1CCOCC1 ; CN(Cc1nc2cc(Cl)ccc2[nH]1)C(=O)C(c1ccc(F)cc1)N1CCCCC1]
or a pharmaceutically acceptable salt thereof.

2. The method of claim 1, wherein the cancerous disease is a brain cancer.

3. The method of claim 2, wherein the brain cancer is a brain metastasis.

4. The method of claim 3, wherein the brain metastasis is a breast cancer brain metastatic tumor.

5. The method of claim 2, wherein the compound crosses the blood-brain barrier.

6. The method of claim 1, wherein the cancerous disease is breast cancer.

7. The method of claim 1, wherein the compound inhibits and/or inactivates acyl-coenzyme A synthetase short-family member 2 (ACSS-2).

8. The method of claim 1, wherein growth of the cancerous disease is inhibited.

9. The method of claim 1, further comprising administering radiation therapy to the subject.

10. The method of claim 9, wherein the radiation therapy is directed to a tumor.

11. A method of inhibiting and/or inactivating acyl-coenzyme A synthetase short-family member 2 (ACSS-2), the method comprising contacting the ACSS-2 with of any one or more of the following compounds: 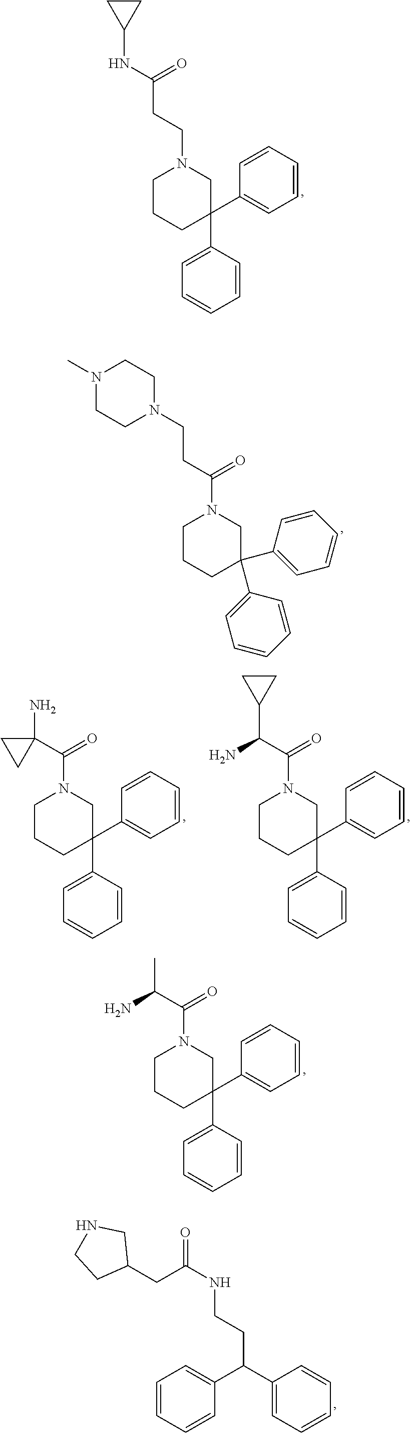 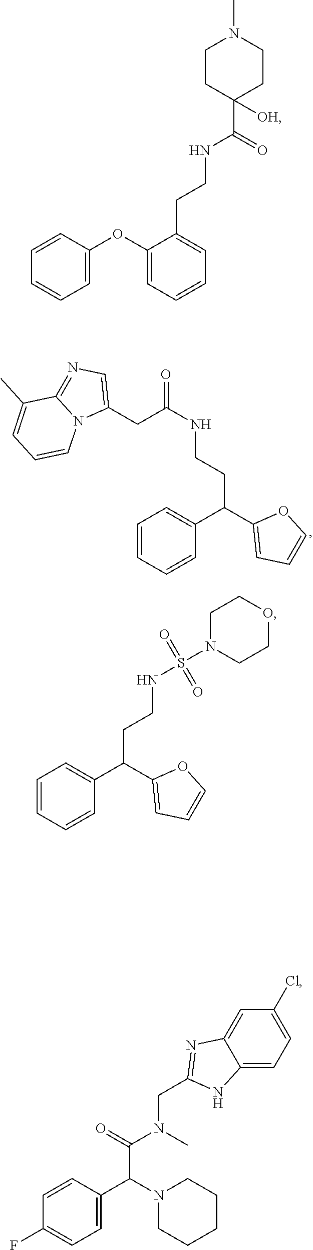
or a pharmaceutically acceptable salt thereof.

12. The method of claim 11, wherein the inhibiting and/or inactivating is done in vitro.

13. The method of claim 11, wherein the inhibiting and/or inactivating comprises administering the one or more compounds to a subject.

14. The method of claim 13, wherein the inhibiting and/or inactivating results in inhibited growth of a cancerous disease in the subject.
